(19) United States
(12) Patent Application Publication
(10) Pub. No.: US 2026/0157850 A1
(43) Pub. Date: Jun. 11, 2026

(54) DELIVERY APPARATUS FOR PROSTHETIC IMPLANTS

(71) Applicant: Edwards Lifesciences Corporation, Irvine, CA (US)

(72) Inventors: Yuanlong Du, Irvine, CA (US); Robert MacCarthy, Irvine, CA (US); Kevin Jiang, Westminster, CA (US); Timothy Lee Humphrey, Anaheim, CA (US)

(21) Appl. No.: 19/537,991

(22) Filed: Feb. 12, 2026

Related U.S. Application Data
(63) Continuation of application No. PCT/US2024/042297, filed on Aug. 14, 2024.
(60) Provisional application No. 63/520,077, filed on Aug. 16, 2023.

(51) Int. Cl. A61F 2/24; A61M 25/10

(52) U.S. Cl. CPC A61F 2/2433; A61M 25/1018

(57) ABSTRACT

Devices and methods for providing fluid to a lumen of a guide catheter with a fluid reservoir fluidly coupled to the lumen, such that pressure within the lumen is maintained at a desired level, are disclosed. As one example, a delivery apparatus can include a handle, a shaft extending within and distally from the handle and having a main lumen, and a reservoir fluidly coupled to the main lumen. The reservoir is filled with fluid and has an adjustable fluid volume, and the reservoir is configured to passively supply fluid to the main lumen based on a fluid pressure in the main lumen.

### CROSS REFERENCE TO RELATED APPLICATION

This application is a continuation of PCT Patent Application No. PCT/US2024/042297 filed on Aug. 14, 2024, which application claims the benefit of U.S. Provisional Patent Application No. 63/520,077 , filed Aug. 16, 2023, each of these applications being hereby is incorporated by reference herein in its entirety.

### FIELD

The present disclosure relates to delivery apparatus for prosthetic implants, including guide catheters through which prosthetic implants and/or their delivery apparatus can be inserted.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing an implant delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The implant delivery apparatus can be inserted through a lumen of the guide catheter to help direct the implant delivery apparatus to a target implantation site (e.g., a native valve region) within the patient and/or help position the implant delivery apparatus at the target implantation site.

### SUMMARY

The foregoing and other objects, features, and advantages of the invention will become more apparent from the following detailed description, which proceeds with reference to the accompanying figures.

Described herein are prosthetic heart valves, docking devices, delivery apparatuses, guide catheters, systems including the guide catheters, and methods for implanting docking devices and prosthetic heart valves. The disclosed guide catheters can, for example, be configured to receive a portion of a delivery apparatus within a main lumen of the guide catheter in order to introduce the delivery apparatus into a patient's vasculature and guide the delivery apparatus toward a target implantation site for a prosthetic medical device mounted on the delivery apparatus. In some examples, a guide catheter system can include a reservoir disposed external to a handle of the guide catheter and configured to passively supply fluid to a main lumen of the guide catheter such that a volume and pressure within the main lumen is maintained. As such, the devices and methods disclosed herein can, among other things, overcome one or more of the deficiencies of typical guide catheters.

A delivery apparatus can comprise a handle and one or more shafts coupled to the handle.

In some examples, the delivery apparatus comprises a reservoir external to the housing.

In some examples, the reservoir is filled with fluid and has an adjustable fluid volume.

In some examples, the delivery apparatus comprises a shaft extending within and distally from the handle and having a main lumen.

In some examples, the main lumen is fluidly coupled to the reservoir by a channel disposed within the handle.

In some examples, the reservoir is configured to passively supply fluid to the main lumen based on a fluid pressure in the main lumen.

In some examples, the reservoir is a compressible reservoir.

In some examples, the reservoir comprises an outer wall that comprises a flexible material, and the reservoir is configured to compress as its fluid volume reduces.

In some examples, the reservoir comprises an inner frame disposed interior of the outer wall, the inner frame defines a compressed volume of the reservoir, and the inner frame is configured to restrict the fluid volume of the reservoir to be at least equal to the compressed volume.

In some examples, the reservoir is configured to transition between an expanded configuration, in which the fluid volume of the reservoir is an expanded volume that is greater than the compressed volume, and a compressed configuration, in which the fluid volume of the reservoir is equal to the compressed volume.

In some examples, the outer wall engages the inner frame when the reservoir is in the compressed configuration.

In some examples, the inner frame is configured to permit the fluid to flow through the inner frame as the reservoir transitions between the expanded configuration and the compressed configuration.

In some examples, the inner frame comprises a rigid material.

In some examples, the handle further comprises a flush port coupled to the housing, and the flush port is fluidly coupled to the reservoir.

In some examples, the reservoir comprises a fluid outlet port, and the delivery apparatus comprises a fluid conduit that fluidly couples the fluid outlet port to the flush port.

In some examples, the fluid conduit terminates at a fluid conduit connector that is configured to engage the flush port to fluidly couple the reservoir to the flush port.

In some examples, the reservoir is a first reservoir, and the delivery apparatus further comprises a second reservoir fluidly connected to the first reservoir.

In some examples, the second reservoir is fluidly connected to the main lumen via the first reservoir.

In some examples, the handle further comprises a flush port coupled to the housing, the reservoir comprises a fluid outlet port, the delivery apparatus comprises a fluid conduit that fluidly couples the fluid outlet port to the flush port with a first connector type, and the second reservoir is coupled to the fluid inlet port with a second connector type that is different from the first connector type.

In some examples, the second reservoir comprises a barrel and a movable wall disposed within the barrel, the barrel and the movable wall defining a second reservoir cavity that contains the fluid therein, and the movable wall is configured to be selectively moved to drive fluid from the second reservoir to the first reservoir.

In some examples, the delivery apparatus comprises a reservoir mount configured to support the reservoir relative to the handle.

In some examples, the reservoir mount is configured to maintain the reservoir in a fixed position relative to the handle.

In some examples, the handle is configured to be supported relative to a delivery apparatus preparation surface by a handle support structure, and the reservoir mount is configured to be coupled to the delivery apparatus preparation surface.

In some examples, the reservoir mount is configured to be one or both of fixedly coupled to the delivery apparatus preparation surface and removably coupled to the delivery apparatus preparation surface.

In some examples, the reservoir mount is configured to be one or both of fixedly coupled to the reservoir and removably coupled to the reservoir.

In some examples, the reservoir is configured to be supported by a delivery apparatus preparation surface at least substantially underneath the handle.

In some examples, the reservoir is at least partially supported by the handle.

In some examples, the reservoir extends at least substantially around a circumference of the handle.

In some examples, the reservoir is fluidly sealed against the handle by one or more reservoir seals.

In some examples, a delivery apparatus comprises one or more of the components recited in examples 1-36 below.

A delivery assembly can comprise an implant catheter, a guide catheter, and a shaft.

In some examples, the guide catheter comprises a handle comprising a housing and a flush port coupled to the housing.

In some examples, the shaft extends distally from within the handle.

In some examples, the shaft has a main lumen configured to receive a portion of the implant catheter therethrough.

In some examples, the delivery apparatus comprises a reservoir disposed external to the housing and filled with fluid.

In some examples, the main lumen is fluidly coupled to the reservoir via the flush port.

In some examples, the reservoir is a compressible reservoir with an adjustable fluid volume.

In some examples, the reservoir comprises an outer wall and an inner frame disposed interior of the outer wall, the inner frame defines a compressed volume of the reservoir, and the inner frame is configured to restrict a fluid volume of the reservoir to be at least equal to the compressed volume.

In some examples, the reservoir is configured to transition between an expanded configuration, in which a fluid volume of the reservoir is an expanded volume that is greater than the compressed volume, and a compressed configuration, in which the fluid volume of the reservoir is equal to the compressed volume.

In some examples, the outer wall engages the inner frame when the reservoir is in the compressed configuration.

In some examples, the inner frame is configured to permit the fluid to flow through the inner frame as the reservoir transitions between the expanded configuration and the compressed configuration.

In some examples, the inner frame comprises a rigid material.

In some examples, the main lumen is fluidly coupled to the reservoir by a channel extending at least partially between the main lumen and the reservoir.

In some examples, the handle further comprises a seal housing assembly including one or more fluid seals, and the sealing housing assembly is disposed at a proximal end of handle.

In some examples, the channel is disposed adjacent to the seal housing assembly.

In some examples, the reservoir comprises a fluid outlet port, and the delivery assembly comprises a fluid conduit that fluidly couples the fluid outlet port to the flush port.

In some examples, the fluid conduit terminates at a fluid conduit connector that is configured to engage the flush port to fluidly couple the reservoir to the flush port.

In some examples, the fluid conduit comprises a flexible conduit.

In some examples, the reservoir is a first reservoir, and the delivery assembly further comprises a second reservoir fluidly connected to the first reservoir.

In some examples, the second reservoir is fluidly connected to the main lumen via the first reservoir.

In some examples, the first reservoir comprises a fluid inlet port, and the second reservoir is coupled to the fluid inlet port.

In some examples, the reservoir comprises a fluid outlet port, the delivery assembly comprises a fluid conduit that fluidly couples the fluid outlet port to the flush port with a first connector type, and the second reservoir is coupled to the fluid inlet port with a second connector type that is different from the first connector type.

In some examples, the second reservoir is fluidly connected to the first reservoir via a stopcock.

In some examples, the second reservoir comprises a barrel and a movable wall disposed within the barrel, the barrel and the movable wall defining a second reservoir cavity that contains the fluid therein, and the movable wall is configured to be selectively moved to drive fluid from the second reservoir to the first reservoir.

In some examples, the delivery assembly further comprises a reservoir mount configured to support the reservoir relative to the handle.

In some examples, the reservoir mount is configured to maintain the reservoir in a fixed position relative to the handle.

In some examples, the handle is configured to be supported relative to a delivery apparatus preparation surface by a handle support structure, and the reservoir mount is configured to be coupled to the delivery apparatus preparation surface.

In some examples, the reservoir mount is configured to be one or both of fixedly coupled to the delivery apparatus preparation surface and removably coupled to the delivery apparatus preparation surface.

In some examples, the reservoir mount is configured to be one or both of fixedly coupled to the reservoir and removably coupled to the reservoir.

In some examples, the reservoir is configured to be supported by a delivery apparatus preparation surface at least substantially underneath the handle.

In some examples, the reservoir is at least partially supported by the handle.

In some examples, the reservoir extends at least substantially around a circumference of the handle.

In some examples, the channel is an annular channel that extends at least substantially circumferentially around the main lumen.

In some examples, the reservoir is fluidly sealed against the handle by one or more reservoir seals.

In some examples, the one or more reservoir seals comprise annular seals positioned on opposite sides of the reservoir.

In some examples, a delivery assembly comprises any of the components recited in examples 37-70 below.

A guide sheath system can comprise a handle and a compressible reservoir disposed external to the housing.

In some examples, the handle comprises a housing and a flush port coupled to the housing.

In some examples, the reservoir is filled with fluid and has an adjustable volume.

In some examples, the flush port is fluidly coupled to the reservoir.

In some examples, the guide sheath system comprises a shaft extending within and distally from the handle and having a main lumen that extends within the housing and through the seal housing assembly.

In some examples, the main lumen is fluidly coupled to the reservoir.

In some examples, the reservoir comprises a flexible wall and a cavity defined by the wall, and the cavity is filled with the fluid.

In some examples, the reservoir comprises an outer wall and an inner frame disposed interior of the outer wall, the inner frame defines a compressed volume of the reservoir, and the inner frame is configured to restrict a fluid volume of the reservoir to be at least equal to the compressed volume.

In some examples, the reservoir is configured to transition between an expanded configuration, in which the fluid volume of the reservoir is an expanded volume that is greater than the compressed volume, and a compressed configuration, in which the fluid volume of the reservoir is equal to the compressed volume.

In some examples, the outer wall engages the inner frame when the reservoir is in the compressed configuration.

In some examples, the inner frame is configured to permit the fluid to flow through the inner frame as the reservoir transitions between the expanded configuration and the compressed configuration.

In some examples, the inner frame comprises a rigid material.

In some examples, the handle comprises a fluid inlet configured as a first channel that extends between the flush port and the main lumen, and the reservoir comprises a fluid outlet port and a fluid conduit configured as a second channel that extends between the fluid outlet port and the fluid inlet.

In some examples, the first channel is disposed adjacent to the seal housing assembly.

In some examples, the reservoir comprises a fluid outlet port, and the guide sheath system comprises a fluid conduit that fluidly couples the fluid outlet port to the flush port.

In some examples, the fluid conduit terminates at a fluid conduit connector that is configured to engage the flush port to fluidly couple the reservoir to the flush port.

In some examples, the fluid conduit comprises a flexible conduit.

In some examples, the reservoir is a first reservoir, and the guide sheath system further comprises a second reservoir fluidly connected to the first reservoir.

In some examples, the second reservoir is fluidly connected to the main lumen via the first reservoir.

In some examples, the first reservoir comprises a fluid inlet port, and the second reservoir is coupled to the fluid inlet port.

In some examples, the first reservoir comprises a fluid outlet port, the guide sheath system comprises a fluid conduit that fluidly couples the fluid outlet port to the flush port with a first connector type, and the second reservoir is coupled to the fluid inlet port with a second connector type that is different from the first connector type.

In some examples, the second reservoir is fluidly connected to the first reservoir via a stopcock.

In some examples, the second reservoir comprises a barrel and a movable wall disposed within the barrel, the barrel and the movable wall defining a second reservoir cavity that contains the fluid therein, and the movable wall is configured to be selectively moved to drive fluid from the second reservoir to the first reservoir.

In some examples, the second reservoir comprises a syringe.

In some examples, the guide sheath system comprises a reservoir mount configured to support the reservoir relative to the handle.

In some examples, the reservoir mount is configured to maintain the reservoir in a fixed position relative to the handle.

In some examples, the handle is configured to be supported relative to a delivery apparatus preparation surface by a handle support structure, and the reservoir mount is configured to be coupled to the delivery apparatus preparation surface.

In some examples, the reservoir mount is configured to be one or both of fixedly coupled to the delivery apparatus preparation surface and removably coupled to the delivery apparatus preparation surface.

In some examples, the reservoir mount is configured to be one or both of fixedly coupled to the reservoir and removably coupled to the reservoir.

In some examples, the reservoir is configured to be supported by a delivery apparatus preparation surface at least substantially underneath the handle.

In some examples, the reservoir is at least partially supported by the handle.

In some examples, the reservoir extends at least substantially around a circumference of the handle.

In some examples, the reservoir is fluidly coupled to the main lumen via an annular channel that extends at least substantially circumferentially around the main lumen.

In some examples, the reservoir is fluidly sealed against the handle by one or more reservoir seals.

In some examples, the one or more reservoir seals comprise annular seals positioned on opposite sides of the reservoir.

In some examples, the handle further comprises a flex mechanism that is configured to adjust a curvature of a distal end portion of the shaft and disposed within the housing, and the flex mechanism is disposed distal to the seal housing assembly.

In some examples, a guide sheath system comprises any of the components recited in examples 71-103 below.

A method for implanting a prosthetic medical device can comprise inserting a shaft of a guide catheter into a vessel of a patient, the shaft having a main lumen and extending from within and distal to a handle of the guide catheter; inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter; and compressing a fluid reservoir disposed external to a housing of the handle and flowing fluid from within the fluid reservoir into the main lumen such that a volume of the fluid reservoir decreases.

In some examples, the compressing the fluid reservoir occurs passively as fluid from within the fluid reservoir is pulled into the main lumen by decreasing fluid pressure within the main lumen as the distal end portion of the first implant catheter is advanced further along the main lumen.

In some examples, the compressing the fluid reservoir includes contracting an external wall of the fluid reservoir inward such that a fluid volume of an internal cavity of the fluid reservoir that contains the fluid decreases as the fluid flows into the main lumen.

In some examples, the fluid reservoir comprises an inner frame disposed interior of the external wall, the inner frame defines a compressed volume of the fluid reservoir, and the inner frame is configured to restrict the fluid volume of the reservoir to be at least equal to the compressed volume.

In some examples, the reservoir is configured to transition between an expanded configuration, in which the fluid volume of the fluid reservoir is an expanded volume that is greater than the compressed volume, and a compressed configuration, in which the fluid volume of the fluid reservoir is equal to the compressed volume.

In some examples, the flowing the fluid from within the fluid reservoir into the main lumen comprises flowing such that the fluid volume of the fluid reservoir reaches the compressed volume.

In some examples, flowing fluid from within the fluid reservoir into the main lumen includes flowing fluid from within the fluid reservoir through a fluid channel extending between the fluid reservoir and the main lumen inside the housing.

In some examples, the fluid reservoir is a first fluid reservoir, and the method further comprises flowing fluid from a second fluid reservoir to the first fluid reservoir to drive fluid from the first fluid reservoir to the main lumen.

In some examples, the first fluid reservoir comprises a fluid inlet port, and the second fluid reservoir is fluidly connected to the fluid inlet port.

In some examples, the second fluid reservoir is connected to the fluid inlet port via a stopcock, and the method further comprises, subsequent to flowing the fluid from the second fluid reservoir to the first fluid reservoir, transitioning the stopcock to a closed position.

In some examples, the method further comprises implanting the prosthetic medical device at the target implantation site, removing the first implant catheter from the guide catheter, and inserting a second implant catheter into the guide catheter and advancing the second implant catheter through the main lumen toward the target implantation site.

In some examples, the first implant catheter is a docking device delivery apparatus and the prosthetic medical device is a docking device, and the second implant catheter is a prosthetic heart valve delivery apparatus configured to deliver a prosthetic heart valve within the implanted docking device.

A method for implanting a prosthetic medical device can comprise inserting a shaft of a guide catheter into a vessel of a patient, the shaft having a main lumen and extending from within and distal to a handle of the guide catheter; and inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter,

In some examples, as the distal end portion of the first implant catheter is advanced through the main lumen, fluid is pulled from a fluid reservoir into the main lumen such that a volume of the fluid reservoir decreases.

In some examples, the fluid reservoir is disposed external to the handle.

In some examples, as fluid is pulled from the fluid reservoir into the main lumen, a wall of the fluid reservoir contracts inward and a fluid cavity containing the fluid and defined by the wall of the fluid reservoir decreases in size.

In some examples, the fluid reservoir is fluidly coupled to the main lumen by a channel disposed within the handle.

In some examples, the handle includes a flush port coupled to a housing of the handle and disposed distal to one or more fluid seals of the handle that are disposed adjacent to the proximal end of the guide catheter, and the fluid reservoir is fluidly coupled to the flush port.

In some examples, the fluid reservoir is a first fluid reservoir, and the method further comprises flowing fluid from a second fluid reservoir to the first fluid reservoir to drive fluid from the first fluid reservoir to the main lumen.

In some examples, the first fluid reservoir comprises a fluid inlet port, and the second fluid reservoir is fluidly connected to the fluid inlet port.

In some examples, the second fluid reservoir is connected to the fluid inlet port via a stopcock, and the method further comprises, subsequent to flowing the fluid from the second fluid reservoir to the first fluid reservoir, transitioning the stopcock to a closed position.

In some examples, the method further comprises implanting the prosthetic medical device at the target implantation site, removing the first implant catheter from the guide catheter, and inserting a second implant catheter into the guide catheter and advancing the second implant catheter through the main lumen toward the target implantation site.

In some examples, the first implant catheter is a docking device delivery apparatus and the prosthetic medical device is a docking device, and the second implant catheter is a prosthetic heart valve delivery apparatus configured to deliver a prosthetic heart valve within the implanted docking device.

In some examples, a method comprises any of the steps of examples 104-124 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a perspective view of an exemplary delivery apparatus for a prosthetic heart valve.

FIG. 6 is side view of an exemplary guide catheter configured to receive a delivery apparatus and guide the delivery apparatus through a portion of a patient's vasculature.

FIG. 7 is a cross-sectional side view of the guide catheter of FIG. 6, according to an example.

FIG. 8 is a side view of a delivery assembly including the guide catheter of FIG. 6 and the delivery apparatus of FIG. 5.

FIG. 9A is a cross-sectional side view of an exemplary guide catheter showing a first adjustable reservoir external to a handle of the guide catheter in an expanded configuration and a second adjustable reservoir external to the handle and fluidly connected to the first reservoir.

FIG. 9B is a cross-sectional side view of the guide catheter of FIG. 9A showing the second adjustable reservoir in a compressed configuration according to an example.

FIG. 9C is a cross-sectional side view of the guide catheter of FIGS. 9A-9B as a delivery apparatus is directed through a main lumen of the guide catheter.

FIG. 9D is a cross-sectional side view of the guide catheter of FIGS. 9A-9C showing the first reservoir in a compressed configuration as the delivery apparatus is directed further along the main lumen of the guide catheter.

FIG. 10 is a perspective view of a guide catheter with a handle supported relative to a delivery apparatus preparation surface and with an external reservoir supported relative to the delivery apparatus preparation surface according to an example.

FIG. 11 is a perspective view of a guide catheter with a handle supported relative to a delivery apparatus preparation surface and with an external reservoir supported relative to the delivery apparatus preparation surface according to another example.

FIG. 12A is a perspective view of a guide catheter with a handle supported relative to a delivery apparatus preparation surface and with an external reservoir supported by the handle according to an example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a steerable delivery apparatus (sometimes referred to as a steerable catheter) that can be used to navigate a subject's vasculature to deliver an implantable, expandable medical device (e.g., a prosthetic heart valve), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

As introduced above, a guide catheter can be inserted into a patient's vasculature and then receive an implant delivery apparatus within a main lumen of the guide catheter in order to direct the delivery apparatus therethrough to a target implantation site for a prosthetic implant. In some examples, an inner diameter of the main lumen of the guide catheter and an outer diameter of portions of the implant delivery apparatus can be closely matched. Thus, in some examples, as the delivery apparatus is pushed distally through the main lumen of the guide catheter, a negative pressure (or vacuum) can be created within the main lumen, proximal to the implant, thereby creating an increase in a pressure gradient across one or more fluid seals within a handle of the guide catheter. This can also result in an increase in a force felt by a user as they push the delivery apparatus through the guide catheter (referred to as “push forces”). Accordingly, improvements to the guide catheter that decrease or prevent negative pressure from being created within the main lumen are desirable. Such improvements can, for example, help maintain hemostasis and/or reduce push forces when advancing a delivery apparatus through the guide catheter.

Described herein are various systems, apparatuses, methods, or the like, that, in some examples, can be used in or with delivery apparatuses for prosthetic medical devices (such as prosthetic heart valves or docking devices). In some examples, such systems, apparatuses, and/or methods can provide a fluid reservoir fluidly coupled to a handle of the guide catheter which is configured to provide fluid to a lumen of the guide catheter (and reduce in volume) and maintain a pressure within the lumen as a delivery apparatus is navigated through the lumen of the guide catheter toward an implantation site in a body of a patient. The fluid reservoir can reduce negative pressure created within the system, thereby reducing a pressure gradient across the fluid seals of the handle of the guide catheter and push forces felt by a user pushing the delivery apparatus through the guide catheter. As a result, hemostasis can be maintained, the system can be easier to operate, and, in some instances, can reduce a likelihood of air being pulled into the guide catheter.

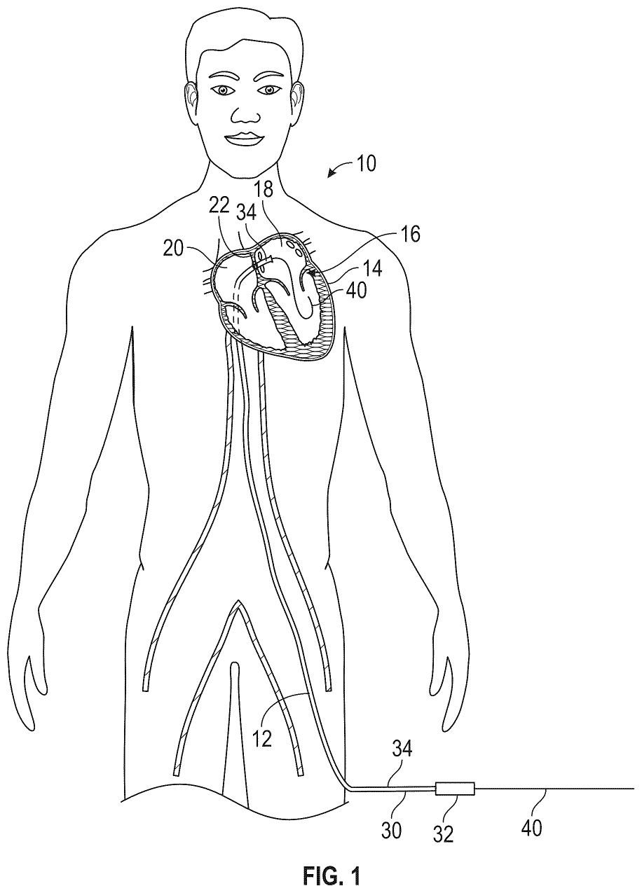

In some examples, the guide catheters disclosed herein can be used to introduce one or more delivery apparatuses (or implant catheters) into the vasculature of a patient and guide the one or more delivery apparatuses at least partially through the vasculature toward a target implantation site. For example, FIGS. 1-4 schematically illustrate an exemplary transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus. The prosthetic heart valve delivery apparatus is used to deliver a transcatheter prosthetic heart valve inside the docking device.

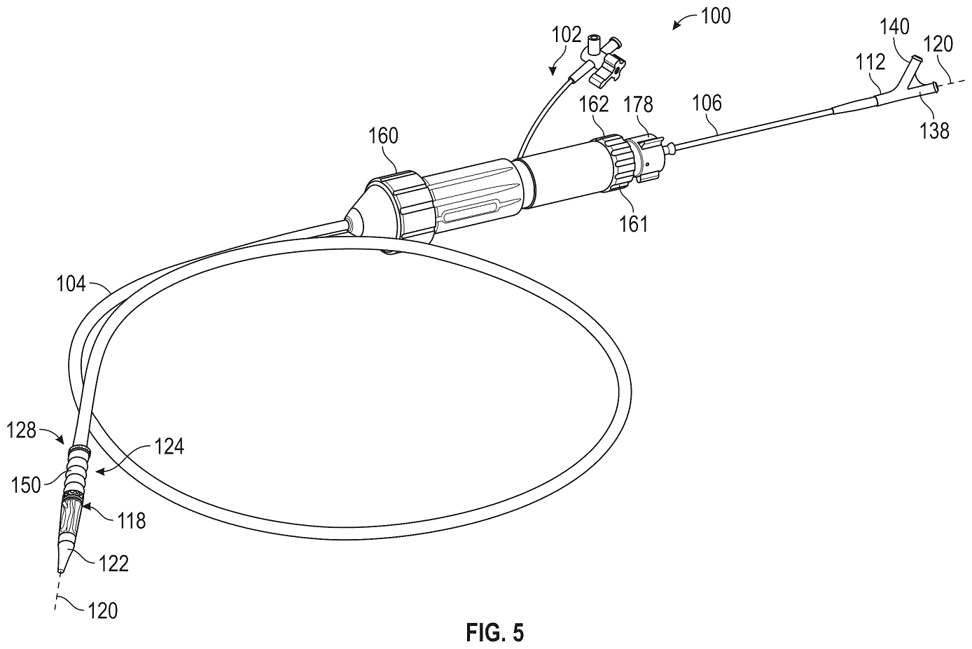

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve. An exemplary delivery apparatus for delivery a prosthetic heart valve within a docking device at a native heart valve is shown in FIG. 5.

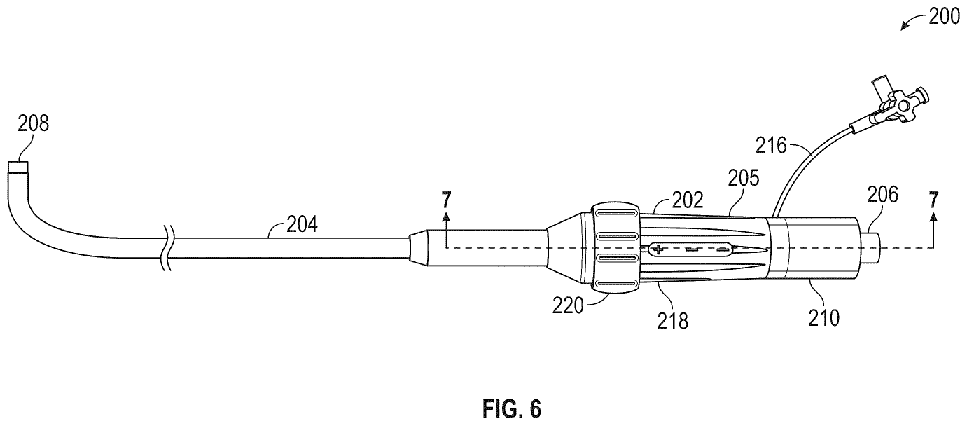

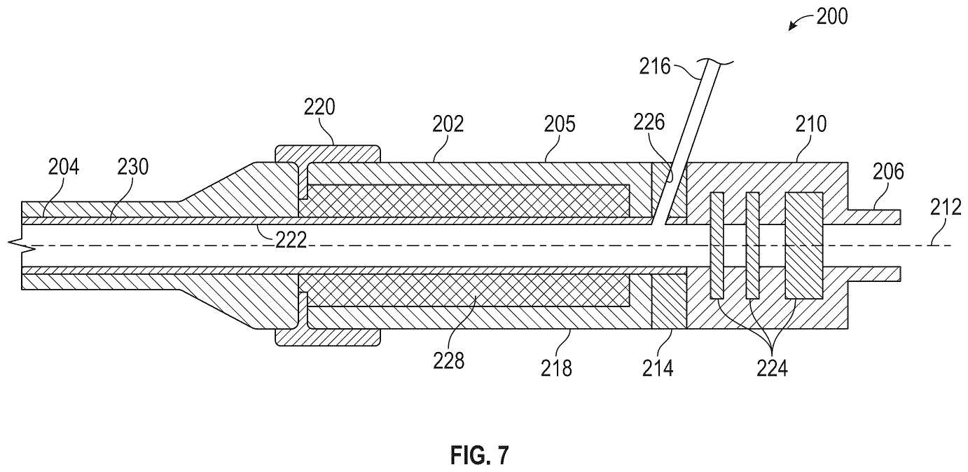

Exemplary guide catheter systems are shown in more detail in FIGS. 6-12B. In some examples, as shown in FIGS. 7A, 7B, and 9A-10B, the guide catheter system can include a reservoir filled with fluid and fluidly coupled to the main lumen of the guide catheter. As such, the reservoir can provide fluid to the main lumen as a delivery apparatus travels through the main lumen. As a result, a pressure gradient across one or more seals within a handle of the guide catheter can be reduced, thereby maintaining hemostasis within the guide catheter, reducing a likelihood of air being introduced into the system (in some instances), and reducing push forces felt by a user operating the delivery apparatus.

### Examples of the Disclosed Technology

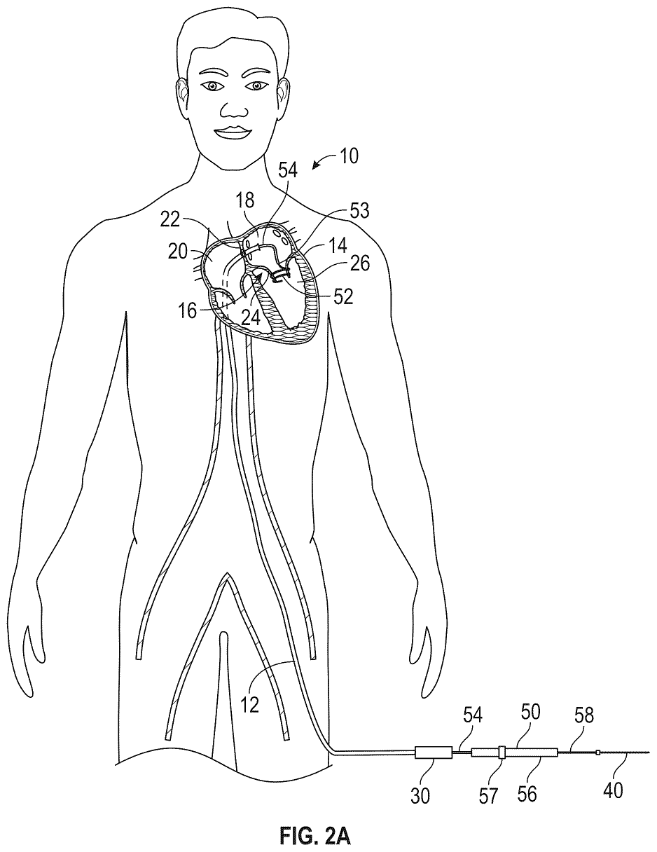

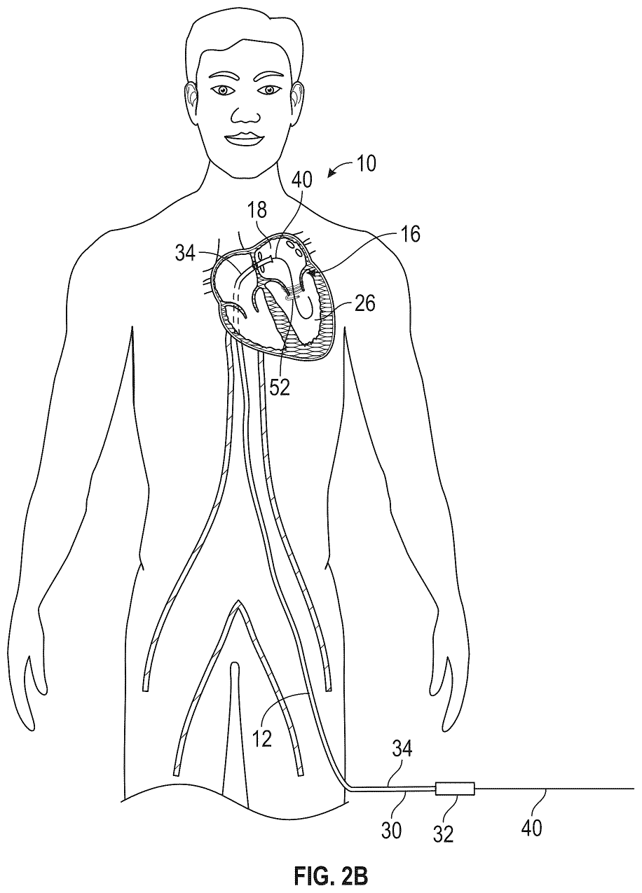

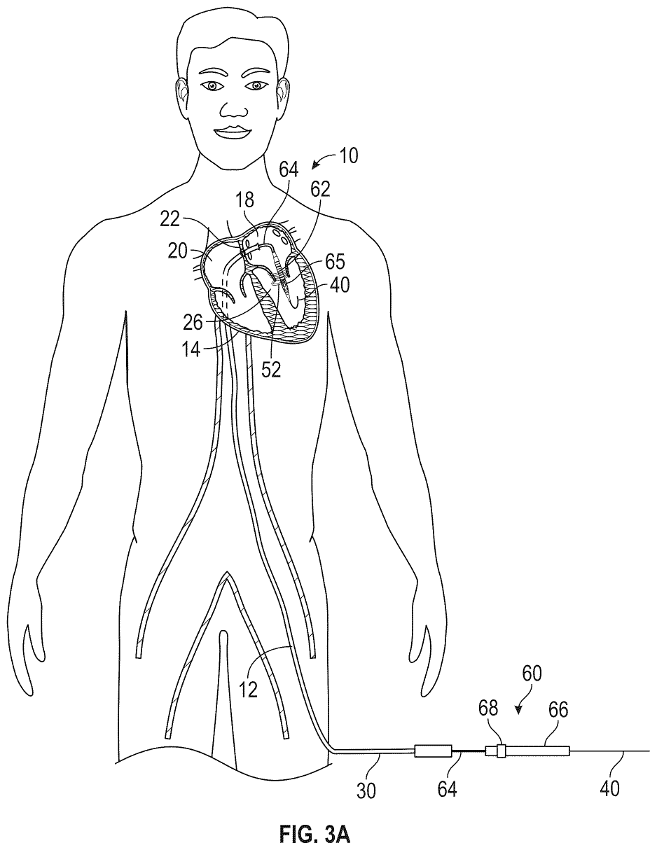

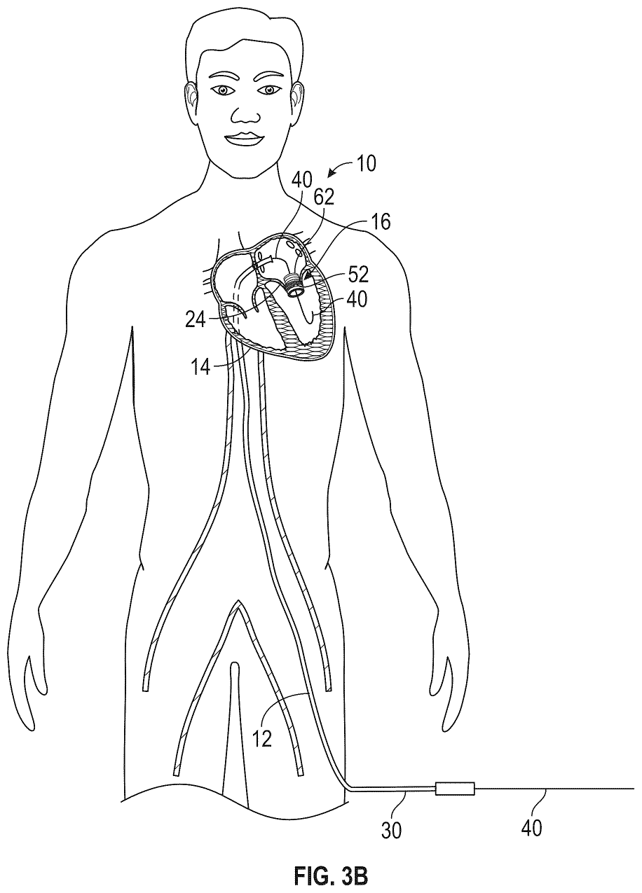

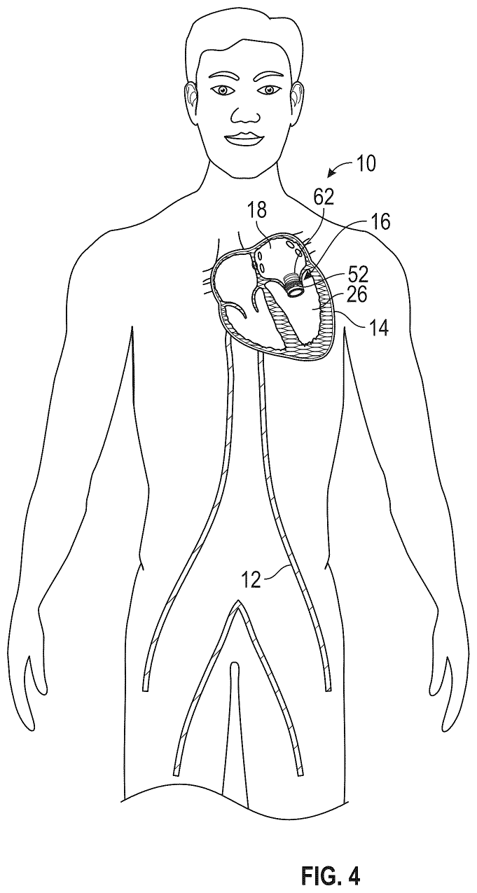

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device”, “introducer”, or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can make a small incision in an atrial septum 22 of the heart 14 to allow access to the left atrium 18 from the right atrium 20. The user can insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In some examples, the prosthetic heart valve 62 can be self-expanding and can be configured to radially expand on its own upon removable of a sheath or capsule covering the radially compressed prosthetic heart valve 62 on the distal end portion of the delivery shaft 64.

In some examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and remove the delivery shaft 54 of the docking device delivery apparatus 50 from the patient 10. The user may advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may expand the prosthetic heart valve 62 within the docking device 52 before removing the prosthetic valve delivery apparatus 60 from the patient 10. In some examples, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and through the aortic valve into the left ventricle 26.

FIG. 5 illustrates an exemplary prosthetic heart valve delivery apparatus 100 (which can also be referred to here as an “implant catheter”) that can be used in lieu of the prosthetic valve delivery apparatus 60 of FIG. 3A to implant an expandable prosthetic heart valve. In some examples, the delivery apparatus 100 is specifically adapted for use in introducing a prosthetic heart valve into a heart.

The delivery apparatus 100 in the illustrated example of FIG. 5 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. In some examples, the delivery apparatus 100 can further comprise an inner shaft extending distally from the handle 102 coaxially through the intermediate shaft 106 and the outer shaft 104 and proximally from the handle 102 coaxially through the intermediate shaft.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion that extends proximally from a proximal end of the handle 102, to an adaptor 112. The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

In some examples, the intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft can extend distally beyond the distal end portion of the intermediate shaft 106 toward or to a nose cone 122 at a distal end of the delivery apparatus 100.

In some examples, a distal end of the balloon 118 can be coupled to a distal end of the delivery apparatus 100, such as to the nose cone 122 (as shown in FIG. 5), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 (shown in FIG. 5) can overly a distal shoulder of the delivery apparatus 100. As shown in FIG. 5, a prosthetic heart valve 150 can be mounted around the balloon 118, at the valve mounting portion 124 of the delivery apparatus 100, in a radially compressed state. The prosthetic heart valve 150 can be configured to be radially expanded by inflation of the balloon 118 at a native valve annulus, as described above with reference to FIGS. 3A and 3B.

A balloon shoulder assembly of the delivery apparatus 100, which includes the distal shoulder, is configured to maintain the prosthetic heart valve 150 (or other medical device) at a fixed position on the balloon 118 during delivery through the patient's vasculature.

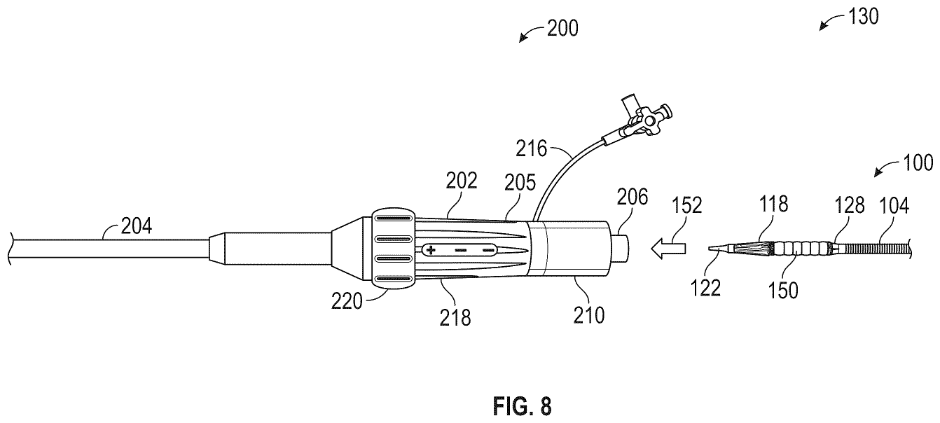

The outer shaft 104 can include a distal tip portion 128 (best seen in FIG. 8) mounted on its distal end. In some examples, the outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 5) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site).

Turning now to FIGS. 6-7, an exemplary guide catheter, which is referred to below as a guide sheath 200 (and can also be referred to herein as a “delivery apparatus” or an “introducer device” or an “introducer”) is shown. In some examples, the guide sheath 200 can be used in lieu of the guide catheter 30 in a docking device and/or a prosthetic valve implantation procedure, as described above with reference to FIGS. 1-4. The guide sheath 200 can be configured to be inserted into a patient's vasculature and receive an implant catheter or delivery apparatus therein (e.g., such as the delivery apparatus 100 of FIG. 5, as shown in FIG. 8) in order to introduce the implant catheter into the patient's vasculature and at least partially guide the implant catheter therein to a target implantations site. Though the guide sheath 200 is described herein as being used with the delivery apparatus 100, the guide sheath 200 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 200 in the illustrated example comprises a handle 202, an elongated shaft 204 extending distally from the handle 202, and a central longitudinal axis 212 (FIG. 7). The shaft 204 has a main (or primary) lumen 222 that is defined by an inner surface of a wall 230 of the shaft 204 (FIG. 7). The main lumen 222 is configured to receive a delivery apparatus therein (such as any of the prosthetic device delivery apparatuses or implant catheters described herein). In some examples, as shown in FIG. 7, the shaft 204 can extend into the handle 202. Further, in some examples, the main lumen 222 can extend through the handle 202 to an inlet port 206 disposed at a proximal end of the handle 202. Thus, in some examples, an inner surface of a wall of a portion of the handle (e.g., at the proximal end) can further define the main lumen 222. Thus, the main lumen 222 can extend from the inlet port 206 to a distal end 208 of the shaft 204.

The handle 202 can have a housing 205 (also referred to as an “outer housing 205”) comprising a main body portion 218 and a seal housing assembly 210 (which can also be referred to as a “seal stack”) which comprises one or more seals 224 contained therein (FIG. 7). The one or more seals 224 of the seal housing assembly 210 can be configured to fluidly seal the main lumen 222 of the guide sheath 200 from the external environment (e.g., from blood, air, and the like). For example, the one or more seals 224 of the seal housing assembly 210 can be configured to prevent blood from a patient in which the guide sheath 200 is inserted from exiting the guide sheath 200 and prevent air from the environment from entering the guide sheath 200 (e.g., through the inlet port 206). The one or more seals 224 can include a variety of types of seals, such as a duckbill seal, a flapper seal, an umbrella valve, a cross-slit valve, a dome valve, or the like.

The handle 202 can, in some instances, include an adaptor spine 214 disposed adjacent and distal to the seal housing assembly 210. The handle 202 can include a flush port 216 connected to the housing 205 at the adaptor spine 214. A flush lumen 226 (or fluid channel) of the adaptor spine 214 is connected to the flush port 216 and further connects to the main lumen 222 (FIG. 7). The flush port 216 can be configured to receive fluid through a lumen thereof. In this way, the flush port 216 can be fluidly coupled to the main lumen 222 by the flush lumen 226.

In some examples, the flush port 216 is connected to the main body portion 218 of the housing 205. While FIGS. 7-8 illustrate the flush port 216 as extending away from the main body portion 218 of the housing 205, it also is within the scope of the present disclosure that the flush port 216 can be directly coupled to and/or at least partially defined by the main body portion 218.

The main body portion 218 is disposed adjacent and distal to the seal housing assembly 210. The handle 202 can include a steering mechanism configured to adjust the curvature of the distal end portion of the shaft 204 (as such, the shaft 204 can be referred to as a steerable shaft). In the illustrated example, the handle 202 includes an adjustment member, such as the illustrated rotatable knob 220 (FIGS. 6-7). The main body portion 218 can house internal flex mechanisms 228 of the guide sheath 200 which are operatively coupled to the rotatable knob 220 (FIG. 7). In some examples, the flex mechanisms 228, and thus the knob 220, can be operatively coupled to the proximal end portion of a pull wire. The pull wire can extend distally from the handle 202 through the shaft 204 and have a distal end portion affixed to the shaft 204 at or near the distal end 208 of the shaft 204. Rotating the knob 220 can increase or decrease the tension in the pull wire, thereby adjusting the curvature of the distal end portion of the shaft 204. Further details on steering or flex mechanisms for a delivery apparatus can be found in U.S. Pat. No. 9,339,384, as already incorporated by reference above.

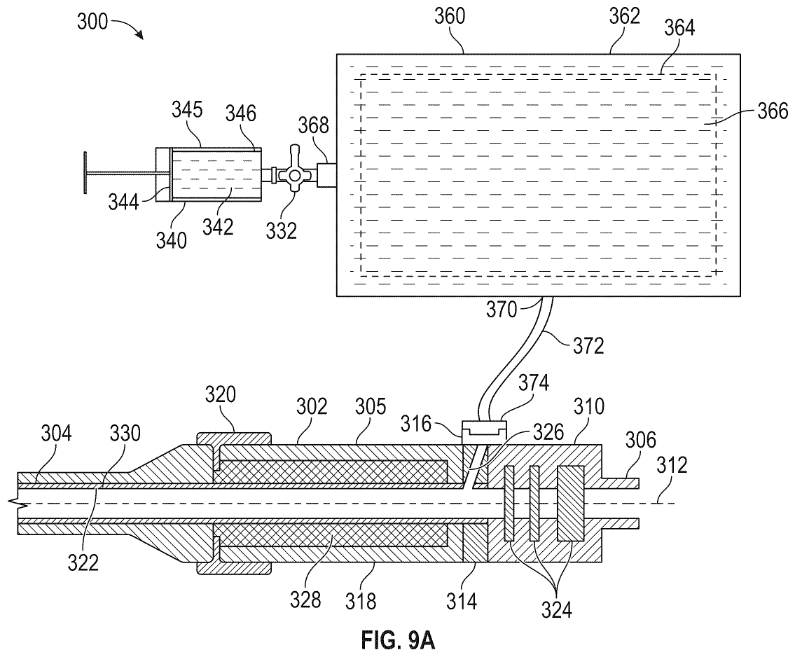

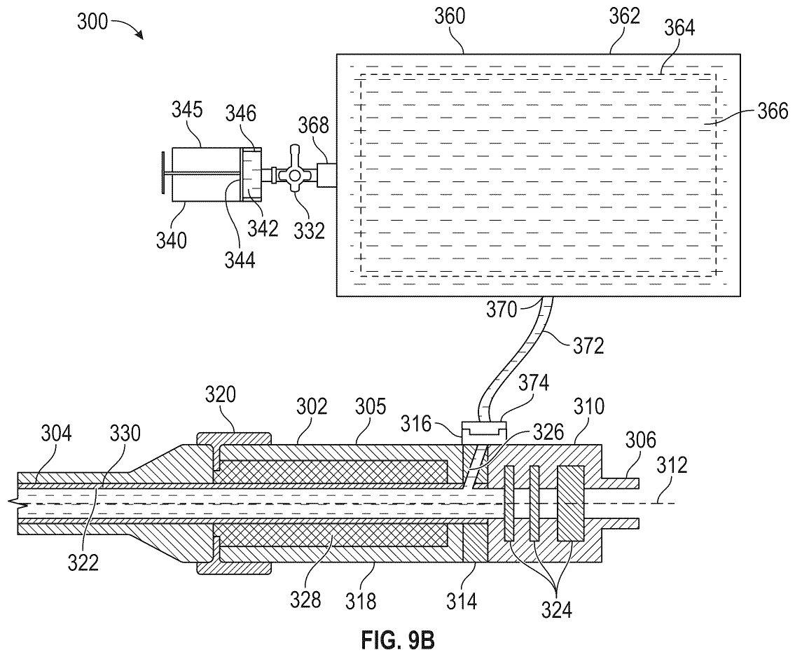

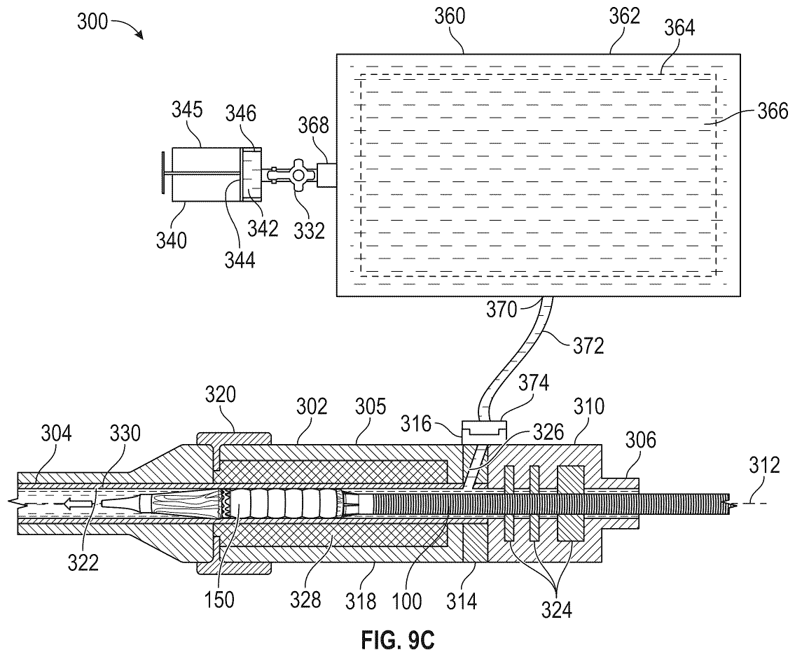

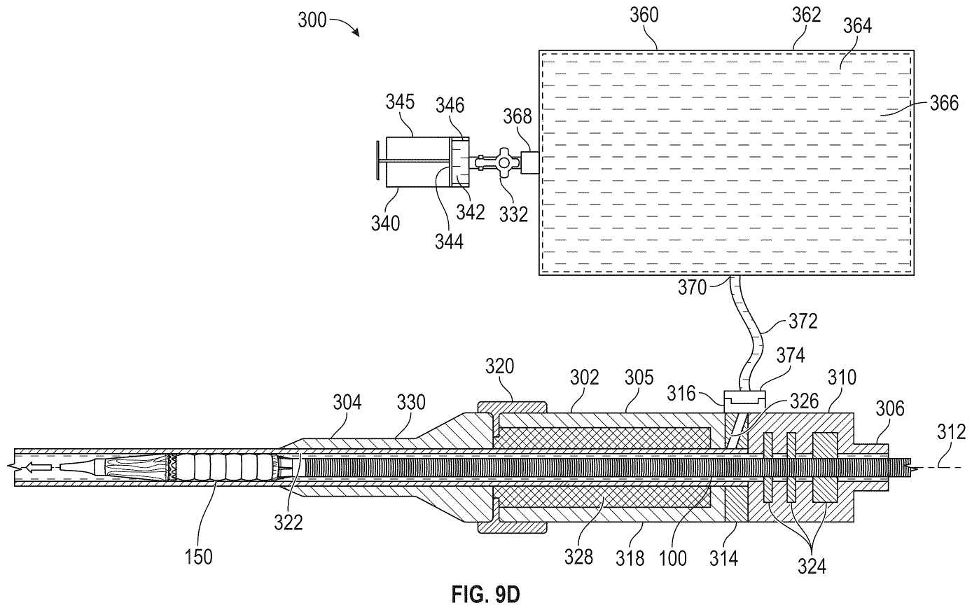

In some examples, a guide sheath similar to the guide sheath 200 of FIGS. 6-8 can be configured to be used in conjunction with, or can include, one or more compressible reservoirs for supplying fluid to the flush port 216. For example, FIGS. 9A-9D depict an exemplary guide catheter or guide sheath 300 (which can also be referred to herein as a “delivery apparatus” or an “introducer device” or an “introducer”). In some examples, the guide sheath 300 can be used in lieu of the guide catheter 30 in a docking device and/or a prosthetic valve implantation procedure, as described above with reference to FIGS. 1-4. The guide sheath 300 can be configured to be inserted into a patient's vasculature and receive an implant catheter or delivery apparatus therein (e.g., such as the delivery apparatus 100 of FIG. 5) in order to introduce the implant catheter into the patient's vasculature and at least partially guide the implant catheter therein to a target implantation site. For example, in some instances, the guide sheath 300 can be used in lieu of the guide sheath 200 of FIGS. 6-8.

Although the guide sheath 300 is described herein as being used with the delivery apparatus 100, the guide sheath 300 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 300 can be similar to the guide sheath 200 except it comprises a reservoir 360 that is disposed external to a handle 302 of the guide sheath (and thus can also be referred to herein as an “external reservoir 360”). Additionally, in the example of FIGS. 9A-9D, the handle 302 includes a flush port 316 in the form of a connector that is mounted on and/or supported by the housing 305 at an adapter spine 314.

The guide sheath 300 can comprise a handle 302, an elongated shaft 304 extending distally from the handle 302, and a central longitudinal axis 312. The shaft 304 has a main (or primary) lumen 322 that is defined by an inner surface of a wall 330 of the shaft 304. The main lumen 322 is configured to receive a delivery apparatus therein (such as any of the prosthetic device delivery apparatuses or implant catheters described herein). In some examples, as shown in FIGS. 9A-9D, the shaft 304 can extend into the handle 302. Further, in some examples, the main lumen 322 can extend through the handle 302 to an inlet port 306 disposed at a proximal end of the handle 302. Thus, in some examples, an inner surface of a wall of a portion of the handle (e.g., at the proximal end) can further define the main lumen 322. Thus, the main lumen 322 can extend from the inlet port 306 to a distal end (e.g., distal end 208 shown in FIG. 6) of the shaft 304.

The handle 302 can have a housing 305 (also referred to as an “outer housing 305”) comprising a main body portion 318 and a seal housing assembly 310 (which can also be referred to as a “seal stack”) which comprises one or more seals 324 contained therein. The one or more seals 324 of the seal housing assembly 310 can be configured to fluidly seal the main lumen 322 of the guide sheath 300 from the external environment. For example, the one or more seals 324 of the seal housing assembly 310 can be configured to prevent blood from a patient in which the guide sheath 300 is inserted from exiting the guide sheath 300 and prevent air from the environment from entering the guide sheath 300 (e.g., through the inlet port 306). The one or more seals 324 can include a variety of types of seals, such as a duckbill seal, a flapper seal, an umbrella valve, a cross-slit valve, a dome valve, or the like.

The handle 302 can, in some instances, include an adaptor spine 314 disposed adjacent and distal to the seal housing assembly 310. In some examples, the flush port 316 is connected to the outer housing 305 at the adaptor spine 314. A flush lumen 326 (or fluid channel) of the adaptor spine 314 is connected to the flush port 316 and further connects to the main lumen 322. The flush port 316 can be configured to receive fluid through a lumen thereof. In this way, the flush port 316 can be fluidly coupled to the main lumen 322 by the flush lumen 326.

The handle 302 can include a steering mechanism configured to adjust the curvature of the distal end portion of the shaft 304 (as such, the shaft 304 can be referred to as a steerable shaft). In the illustrated example, the handle 302 includes an adjustment member, such as the illustrated rotatable knob 320. The main body portion 318 can house internal flex mechanisms 328 of the guide sheath 300 which are operatively coupled to the rotatable knob 320. In some examples, the flex mechanisms 328, and thus the knob 320, can be operatively coupled to the proximal end portion of a pull wire. The pull wire can extend distally from the handle 302 through the shaft 304 and have a distal end portion affixed to the shaft 304 at or near the distal end of the shaft 304. Rotating the knob 320 can increase or decrease the tension in the pull wire, thereby adjusting the curvature of the distal end portion of the shaft 304. Further details on steering or flex mechanisms for a delivery apparatus can be found in U.S. Pat. No. 9,339,384.

As shown in FIGS. 9A-9D, the reservoir 360 is disposed external to the housing 305 of the handle 302. The reservoir 360 is filled with a fluid 342 and has an adjustable fluid volume. For example, the reservoir 360 can comprise a wall 362 (e.g., an outer wall and/or an external wall) defining an internal cavity 366 which contains the fluid 342 therein. The wall 362 can comprise a flexible material that is configured to expand (or stretch) with increasing fluid volume (of the fluid 242) within the reservoir 360 and compress (or fold or contract) with decreasing fluid volume within the reservoir 360.

In some instances, the wall 362 can be configured to conform to the volume of the fluid 342 as the volume of fluid 342 changes, thereby changing a size of the cavity 366. In this way, the reservoir 360 can be configured as an expandable and compressible reservoir, bladder, or balloon with an adjustable (or reducible) fluid volume. As described further below, the wall 362 of the reservoir 340 can compress as its internal volume of the fluid 342 decreases, thereby decreasing the overall volume of the cavity 346. In some examples, the fluid 342 used to fill the reservoir 360 and main lumen 322 is saline or an alternate biocompatible flush fluid.

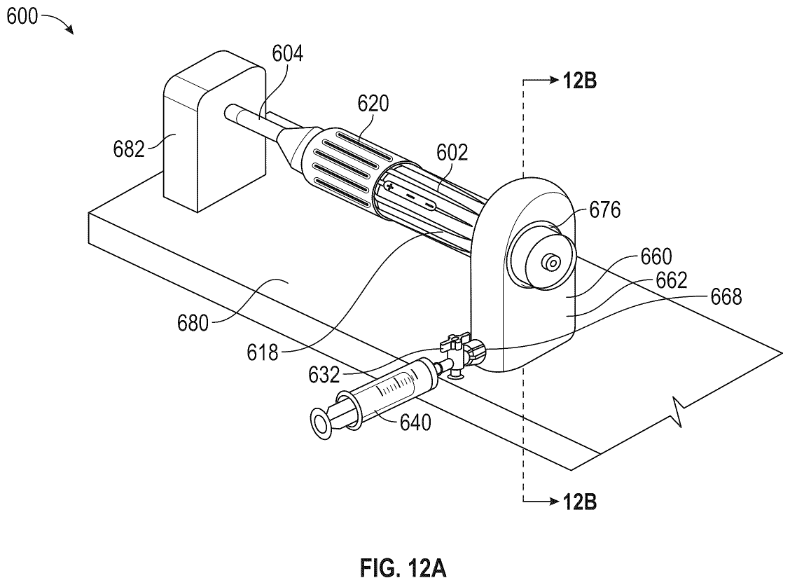

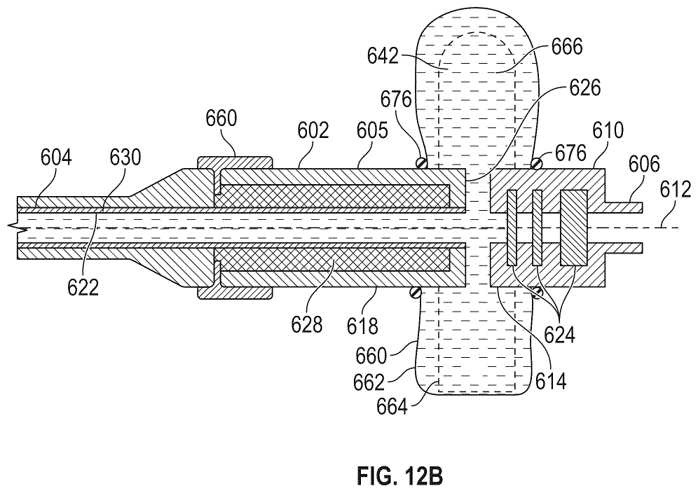

It should be noted that fluid within the main lumen 322 (and the main lumens shown in the other figures, such as FIG. 12B) and the various reservoirs is depicted with a generic hatching. In some examples, however, fluid downstream or distal to the prosthetic heart valve of the delivery apparatus inside the guide sheath can be a combination of priming fluid (e.g., saline) and blood, while fluid upstream of the prosthetic heart valve of the delivery apparatus can be fluid provided from the reservoir (e.g., saline).

The reservoir 360 can be fluidly coupled to the main lumen 322 of the shaft 304 by a channel 326 (or flush lumen) of the handle 302. For example, the channel 326 can extend from the flush port 316, through the housing 305, and to the main lumen 322 such that the main lumen 322 of the shaft 304 is fluidly coupled to the reservoir 360 via the channel 326. The main lumen 322 can be configured to receive fluid from the flush port 316 via the channel 326 (e.g., during preparation or flushing of the guide sheath 300, as described further below). Thus, the channel 326 can serve as and be referred to as a fluid inlet of the main lumen 322.

In some examples, and as shown in FIGS. 9A-9D, the guide sheath system 300 includes a fluid conduit 372 that fluidly interconnects the reservoir 360 and the flush port 316. The fluid conduit 372 can include and/or terminate at a fluid conduit connector 374 that is configured to engage and/or otherwise connect to the flush port 316 to fluidly couple the reservoir 360 to the channel 326. In some examples, the fluid conduit 372 can be a flexible conduit (e.g., a flexible tube) that allows for the reservoir 360 to be selectively positioned relative to the handle 302.

The fluid conduit 372 can extend from and/or at least partially define a fluid outlet port 370 of the reservoir 360. In some examples, the fluid outlet port 370 can be configured to permit the fluid conduit 372 to be selectively removed from the reservoir 360. In other examples, the fluid conduit 372 can be fixedly and/or permanently connected to the reservoir 360 at the fluid outlet port 370.

In some examples, and as shown in FIGS. 9A-9D, the guide sheath system 300 additionally includes a second reservoir 340 fluidly coupled to the main lumen 322 via the reservoir 360. In such examples, the reservoir 360 may be referred to as a first reservoir 360. As shown in FIGS. 9A-9D, the second reservoir 340 also is disposed external to the housing 305 of the handle 302 and is filled with the fluid 342. Similar to the first reservoir 360, the second reservoir 340 has an adjustable fluid volume. For example, the reservoir 340 can comprise a wall 344 defining a second reservoir internal cavity 346 that contains the fluid 342 therein. In particular, in the example of FIGS. 9A-9D, the second reservoir 340 includes a barrel 345 and a movable wall 344 (also referred to as a plunger) disposed within the barrel 345. The barrel 345 and the movable wall 344 at least partially define the second reservoir internal cavity 346 that contains the fluid 342 therein.

In some examples, the second reservoir 340 includes and/or is a syringe.

The first reservoir 360 can include a fluid inlet port 368 for receiving the fluid 342 from the second reservoir 340. In particular, and as shown in FIGS. 9A-9D, the second reservoir 340 can be coupled to the fluid inlet port 368 via a stopcock 332 or another type of connector or adapter that can open and close the fluid connection between the second reservoir 340 and the first reservoir 360. For example, when the stopcock 332 is in the open position, as shown in FIGS. 9A and 9B, fluid can be transferred (and flow) from the second reservoir 340 to the first reservoir 360 via the fluid inlet port 368, and can further be transferred (and flow) from the first reservoir 360 to the main lumen 322 via the fluid outlet port 370, the fluid conduit 372, the flush port 316, and/or the channel 326.

In some examples, the second reservoir 360 can be directly coupled to the fluid inlet port 368.

In some examples, the first reservoir 360 can be fluidly connected to the flush port 316 at a connector type that is different than a connector type at which the second reservoir 340 is fluidly connected to the first reservoir 360. For example, the fluid conduit connector 374 can be configured to be connected to the flush port 316 with a first connector type, and the second reservoir 360 can be configured to be connected to the fluid inlet port 368 at a second connector type that is different from the first connector type. As a more specific example, the second connector type can be a standardized connector type, such as a luer lock connector and/or a threaded coupling. In some examples, the first connector type can be a non-standard connector type, such as a proprietary connector and/or a connector that is unique to the guide sheath system 300. In this manner, the guide sheath system 300 can be configured to restrict and/or prevent a user from inadvertently connecting the second reservoir 360 directly to the flush port 316, instead ensuring that the first reservoir 360 be positioned between the second reservoir 340 and the handle 302 prior to flushing the main lumen 322 with the fluid 342 as described herein.

In some examples, and as shown in FIGS. 9A-9D, the reservoir 360 includes an inner frame 364 disposed interior of the wall 362. The inner frame 364 can be configured to limit an extent to which the wall 362 can contract. For example, the inner frame 364 can define a compressed volume of the cavity 366 such that the fluid volume of the reservoir 360 is restricted to be equal to or greater than the compressed volume. Accordingly, the reservoir 360 can be configured to transition between an expanded configuration (FIGS. 9A-9C), in which the fluid volume of the fluid 342 within the reservoir 360 is an expanded fluid volume, and a compressed configuration (FIG. 9D), in which the fluid volume is equal to the compressed volume, which is less than the expanded fluid volume. The expanded volume also may be referred to as a first volume, and/or the compressed volume also may be referred to as a second volume.

In some examples, the wall 362 engages the inner frame 364 when the reservoir 360 is in the compressed configuration. For example, the inner frame 364 can be formed of a rigid material (e.g., a plastic or a metal) that restricts and/or prevents further contraction of the wall 362 when the wall 362 engages the inner frame 364. The inner frame 364 can be configured to permit the fluid 342 to flow through the inner frame 364 as the reservoir 360 transitions between the expanded configuration and the compressed configuration. For example, the inner frame 364 can include and/or be a mesh structure and/or a perforated structure. Additionally or alternatively, the inner frame 364 can include a plurality of spaced-apart frame members that permit a flow of the fluid 342 therebetween.

As introduced above, the guide sheath 300 can be configured to receive a delivery apparatus, such as the delivery apparatus 100 of FIG. 5, within the main lumen 322 of the guide sheath 300. Prior to inserting the delivery apparatus 100 into the guide sheath (and/or prior to inserting the guide sheath into the vasculature of the patient), the main lumen 322 can be primed or flushed through the flush port 316. For example, fluid (e.g., fluid 342) can flow from the cavity 366 of the first reservoir 360 and/or from the second reservoir cavity 346 of the second reservoir 360 through the flush port 316 and into the main lumen 322 via the channel 326. Subsequent to flowing the fluid 342 through the flush port 316 and into the main lumen 322, the delivery apparatus 100 can be inserted into the guide sheath 300. FIGS. 9A-9D depict different exemplary states of such a process.

After positioning the shaft 304 of the guide sheath 300 within the vasculature of a patient, a distal end portion of the delivery apparatus 100 (e.g., the nose cone 122 and radially compressed prosthetic heart valve 150) can be inserted into the inlet port 306 of the handle 302 of the guide sheath 300, as indicated by arrow 152 in FIG. 8. The distal end portion of the delivery apparatus 100 can be navigated through the seal housing assembly 310 and into the main lumen 322 of the guide sheath 300. The delivery apparatus 100 can continue to be navigated through the main lumen 322 of the shaft 304, toward the implantation site. The assembly shown in FIG. 8 can be referred to as a delivery assembly 130.

FIG. 9A depicts a state in which each of the first reservoir 360 and the second reservoir 340 is fluidly connected to the main lumen 322 via the fluid conduit 372 prior to flowing the fluid 342 into the main lumen. In this state, the first reservoir 360 is at or near the expanded configuration, and the stopcock 332 is in the open position.

In some instances, as the movable wall 344 of the second reservoir 340 is pushed into the barrel 345, fluid exits the second reservoir 340 and the fluid volume of the fluid 342 in the second reservoir 340 decreases. For example, as shown in FIG. 9B, depressing the movable wall 344 of the second reservoir 360 can reduce the fluid volume of the second reservoir cavity 346, thus driving the fluid 342 into the first reservoir 360. In this way, the second reservoir 340 can be configured as an expandable and contractable (or reducible) reservoir (via movement of the movable wall 344) with an adjustable (or reducible) fluid volume.

Upon receiving the fluid 342 from the second reservoir 340, the wall 362 of first reservoir 360 may expand slightly (e.g., to approach and/or reach the expanded configuration) under this increase in fluid pressure. The fluid 342 in the first reservoir 360 correspondingly can be driven through the fluid outlet port 370 and the fluid conduit 372 into the main lumen 322 via the flush port 316 and the channel 326, thereby flushing the main lumen 322. As shown in FIG. 9B, the seals 324 can restrict the fluid 342 to a region of the main lumen 322 distal to the seal housing assembly 310.

With the main lumen 322 flushed with fluid 342 and thus prepared to receive the delivery apparatus 100, the stopcock 332 can be moved to the closed position (FIG. 9C), and the delivery apparatus 100 can be inserted into the main lumen 322 via the seal housing assembly 310. In the configuration of FIG. 9C, the distal end portion of the delivery apparatus 100, including the prosthetic heart valve 150, has just been inserted into the main lumen 322 and is disposed within the handle 302.

As the distal end portion of the delivery apparatus 100 is navigated further through the main lumen 322, pressure (fluid pressure) in a proximal portion of the main lumen 322 (e.g., the portion within the handle 302 and shown in FIG. 9C) can decrease due to the prosthetic heart valve 150 (or alternate portions of the delivery apparatus 100) sliding against the inner surface of the wall 330 of the shaft 304. As the pressure within the main lumen 322 decreases, the fluid 342 can be pulled from the cavity 366 of the first reservoir 360 into the main lumen 322. As a result, the wall 362 of the first reservoir 360 compresses inward toward the inner frame 364, thereby decreasing the internal volume of the cavity 366 (as shown in FIG. 9D). As a result, the pressure within the main lumen 322 can equalize, and in some instances, a negative pressure (or vacuum) created by advancing the distal end portion of the delivery apparatus 100 through the main lumen 322 can be reduced. In this way, the first reservoir 360 can be configured as an expandable and contractable (or reducible) reservoir (via movement of the outer wall 362) with an adjustable (or reducible) fluid volume.

In some examples, the stopcock 332 is closed during this process such that no fluid is enters the first reservoir 360 via the fluid inlet port 368.

In some examples, flowing the fluid 342 from the first reservoir 360 into the main lumen 322 as a result of the decreased pressure within the main lumen 322 can limit an extent to which fluid and/or air is introduced into the main lumen 322 via the seals 324 due to such a pressure difference. In the event that air is introduced into the main lumen 322 (e.g., during insertion of the delivery apparatus 100 into the main lumen 322, the first reservoir 360 and/or the second reservoir 340 may be used to aspirate the air from the main lumen 322. For instance, in some examples, the flush port 316 may be positioned on an upper side of the handle 302 such that any air that is introduced into the main lumen 322 can be passively drawn into the first reservoir 360 via the channel 326 and/or the fluid conduit 327. In some examples, such air can then be removed from the first reservoir 360. For example, the air can be drawn into the second reservoir 340, such as by opening the stopcock 332 and pulling the movable wall 344 of the second reservoir 340 outward to draw the air into the second reservoir 340. Additionally or alternatively, the air can be removed from the first reservoir 360 by uncoupling the second reservoir 340 from the first reservoir 360 and expelling the air through the fluid inlet 368. Accordingly, in some examples, the fluid inlet 368 of the first reservoir 360 may be positioned on an upper end of the cavity 366 to allow for air to collect near and/or be removed from the fluid inlet 368.

In the expanded state of the reservoir 360 (FIGS. 9A-9C), the cavity 366 has the first (expanded) fluid volume. In the compressed state or configuration of the reservoir 360 (FIG. 9D), the cavity 366 has the second (compressed) fluid volume. As shown in FIG. 9D, the compressed configuration of the reservoir 360 can correspond to a configuration in which the wall 362 engages, extends adjacent to, and/or conforms to the inner frame 364 of the first reservoir 360.

In some instances, a size (e.g., diameter, width, and/or length) of the channel 326 can be specified based on a selected fluid volume transfer rate that maintains pressure within the main lumen 322 at a desired level (e.g., a non-negative pressure) and maintains hemostasis within the guide sheath. For example, by increasing a volume of the channel 326, the fluid transfer rate between the reservoir 360 and the main lumen 322 can increase, thereby better maintaining the pressure within the main lumen 322 at a non-negative value as the delivery apparatus is navigated through the main lumen 322.

In some examples, by maintaining the pressure within the main lumen of the guide sheath or catheter at a non-negative level by using the compressible reservoir, push forces felt by a user navigating the delivery apparatus through the main lumen of the guide catheter can also be reduced.

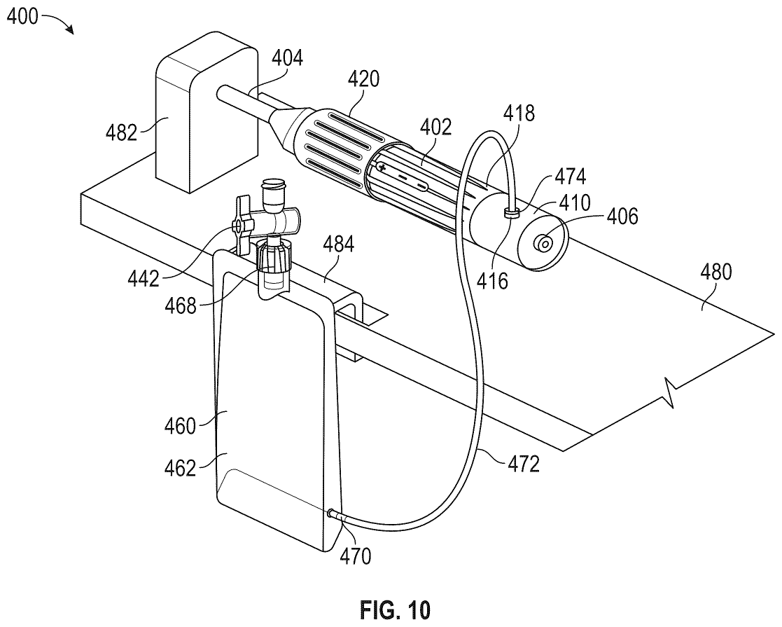

FIG. 10 depicts an exemplary guide catheter or guide sheath 400 (which can also be referred to herein as a “delivery apparatus” or an “introducer device” or an “introducer”). In some examples, the guide sheath 400 can be used in lieu of the guide catheter 30 in a docking device and/or a prosthetic valve implantation procedure, as described above with reference to FIGS. 1-4. The guide sheath 400 can be configured to be inserted into a patient's vasculature and receive an implant catheter or delivery apparatus therein (e.g., such as the delivery apparatus 100 of FIG. 5) in order to introduce the implant catheter into the patient's vasculature and at least partially guide the implant catheter therein to a target implantation site. For example, in some instances, the guide sheath 400 can be used in lieu of the guide sheath 200 of FIGS. 6-8 and/or the guide sheath 300 of FIGS. 9A-9D.

Although the guide sheath 400 is described herein as being used with the delivery apparatus 100, the guide sheath 400 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 400 can be similar to the guide sheath 300. For example, the guide sheath 400 can comprise a handle 402 and an elongated shaft 404 extending distally from the handle 402.

The handle 402 can have a housing 405 (also referred to as an “outer housing 405”) comprising a main body portion 418 and a seal housing assembly 410 (which can also be referred to as a “seal stack”) which comprises one or more seals contained therein (e.g., the seals 324 of FIGS. 9A-9D).

A flush port 416 can be connected to the outer housing 405. For example, the flush port 416 can include and/or be a connector mounted on and/or supported by the housing 405. The flush port 416 is fluidly coupled to a fluid outlet port 470 of a reservoir 460 via a fluid conduit 472. The reservoir 460 includes a fluid inlet port 468 that is connected to a stopcock 432 for receiving fluid from a second reservoir in the manner discussed above with reference to FIGS. 9A-9D. The guide sheath 400 additionally can include any suitable components and/or features discussed above in the context of the guide sheath 200 and/or the guide sheath 300.

In the example of FIG. 10, the handle 402 is supported relative to a delivery apparatus preparation surface 480 by a handle support structure 482. In particular, in the example of FIG. 10, the handle support structure 482 extends around and engages the shaft 404 to support the handle 402 above the delivery apparatus preparation surface 480.

Additionally, in the example of FIG. 10, the reservoir 460 is supported relative to the handle 402 by a reservoir mount 484 that coupled (e.g., fixedly coupled) to the delivery apparatus preparation surface 480. As shown in FIG. 10, the reservoir mount 484 can support the reservoir 460 such that the reservoir 460 is suspended from an edge of the delivery apparatus preparation surface 480. In some examples, the reservoir mount 484 can be removably coupled to the delivery apparatus preparation surface 480 and/or to the reservoir 460.

In the example of FIG. 10, the reservoir 460 is supported by the reservoir mount 484 such that the fluid inlet 468 of the reservoir 460 is at an upper end of the reservoir 460. As discussed above, such a configuration may facilitate removal and/or aspiration of air that enters the reservoir 460 from the handle 402.

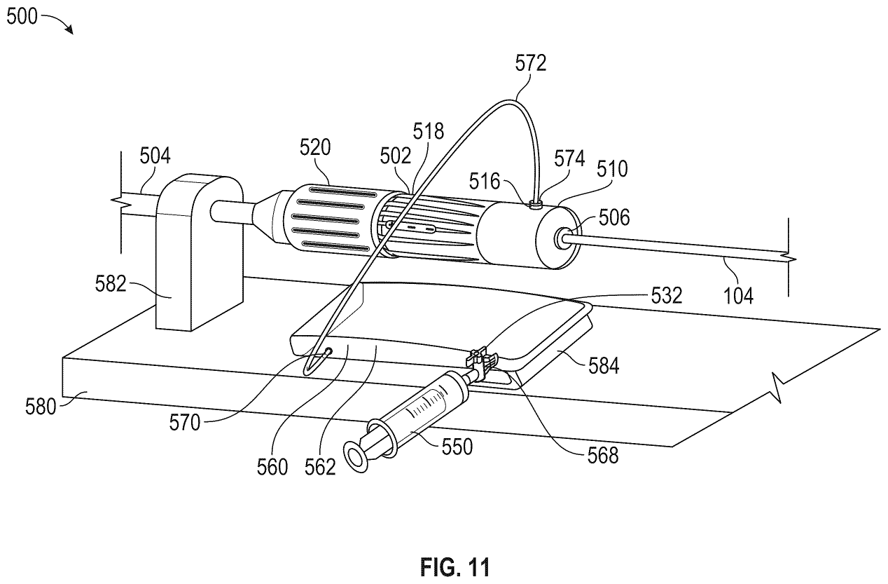

FIG. 11 depicts another exemplary guide catheter or guide sheath 500 (which can also be referred to herein as a “delivery apparatus” or an “introducer device” or an “introducer”). In some examples, the guide sheath 500 can be used in lieu of the guide catheter 30 in a docking device and/or a prosthetic valve implantation procedure, as described above with reference to FIGS. 1-4. The guide sheath 500 can be configured to be inserted into a patient's vasculature and receive an implant catheter or delivery apparatus therein (e.g., such as the delivery apparatus 100 of FIG. 5) in order to introduce the implant catheter into the patient's vasculature and at least partially guide the implant catheter therein to a target implantation site. For example, in some instances, the guide sheath 500 can be used in lieu of the guide sheath 200 of FIGS. 6-8, the guide sheath 300 of FIGS. 9A-9D, and/or the guide sheath 400 of FIG. 10.

Although the guide sheath 500 is described herein as being used with the delivery apparatus 100, the guide sheath 500 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 500 can be similar to the guide sheath 300 and/or the guide sheath 400. For example, the guide sheath 500 can comprise a handle 502 and an elongated shaft 504 extending distally from the handle 502. Similar to the guide sheath 400 of FIG. 10, the handle 502 of the guide sheath 500 is supported relative to a delivery apparatus preparation surface 580 by a handle support structure 582. In particular, in the example of FIG. 11, the handle support structure 582 extends around and engages the shaft 504 to support the handle 502 above the delivery apparatus preparation surface 580.

Additionally, in the example of FIG. 11, the reservoir 560 is supported relative to the handle 502 by a reservoir mount 584 that coupled (e.g., fixedly coupled) to an upper surface of the delivery apparatus preparation surface 580. As shown in FIG. 11, the reservoir mount 584 can support the reservoir 560 such that the reservoir 560 is supported by the delivery apparatus preparation surface 580, such as below the handle 502.

In some examples, the reservoir mount 584 can be removably coupled to the delivery apparatus preparation surface 580 and/or to the reservoir 560. In other examples, the reservoir 560 can be supported by the delivery apparatus preparation surface 580 without the use of a reservoir mount 584.

FIG. 11 additionally shows a configuration in which a second reservoir 540 is coupled to the fluid inlet port 568 of the (first) reservoir 560 via the stopcock 532, and in which the outer shaft 104 of a delivery apparatus (e.g., the delivery apparatus 100 of FIG. 5) extends through the handle 502 and the shaft 504.

FIGS. 12A-12B depict another exemplary guide catheter or guide sheath 600 (which can also be referred to herein as a “delivery apparatus” or an “introducer device” or an “introducer”). In some examples, the guide sheath 600 can be used in lieu of the guide catheter 30 in a docking device and/or a prosthetic valve implantation procedure, as described above with reference to FIGS. 1-4. The guide sheath 600 can be configured to be inserted into a patient's vasculature and receive an implant catheter or delivery apparatus therein (e.g., such as the delivery apparatus 100 of FIG. 5) in order to introduce the implant catheter into the patient's vasculature and at least partially guide the implant catheter therein to a target implantation site. For example, in some instances, the guide sheath 600 can be used in lieu of the guide sheath 200 of FIGS. 6-8, the guide sheath 300 of FIGS. 9A-9D, the guide sheath 400 of FIG. 10, and/or the guide sheath 500 of FIG. 11.

Although the guide sheath 600 is described herein as being used with the delivery apparatus 100, the guide sheath 600 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

The guide sheath 600 can be similar to the guide sheath 300, the guide sheath 400, and/or the guide sheath 500. For example, the guide sheath 600 can comprise a handle 602 and an elongated shaft 604 extending distally from the handle 602. Unlike the guide sheath 400 and the guide sheath 500, however, the guide sheath 600 includes a first reservoir 660 that extends around the handle 602. Specifically, and as shown in FIG. 12A, the first reservoir 660 extends fully around a circumference of the handle 602, and thus may be at least partially supported by the handle 602. Additionally or alternatively, the first reservoir 660 can contact the delivery apparatus preparation surface 680 such that the first reservoir 660 is at least partially supported by the delivery apparatus preparation surface 680.

The first reservoir 660 can be fluidly coupled to the handle 602 in any suitable manner. For example, and as shown in FIG. 12B, the first reservoir 660 can include an outer wall 662 that at least partially defines an inner cavity 666 that contains a volume of fluid 642. The first reservoir 660 can be open to and/or sealed against an outer housing 605 of the handle 602, such as by one or more reservoir seals 676. In the example of FIGS. 12A-12B, the reservoir seals 676 take the form of annular seals (e.g., O-rings and/or gaskets) extending around the handle 602. It is to be understood, however, that any other form of sealing structure may be used to form a fluid-tight seal between the first reservoir 660 and the handle 602.

As shown in FIG. 12B, the handle 602 can include and/or define a channel 226 that fluidly interconnects the inner cavity 666 of the first reservoir 660 and the main lumen 622 of the shaft 204. In the example of FIG. 12B, the channel 626 is an annular (or at least substantially annular) channel that extends at least substantially circumferentially around the main lumen 622. In such examples, the handle 602 can include any of a variety of components and/or structures that interconnect and/or fixedly couple together portions of the handle 602 distal to and proximal to the channel 626. In other examples, the channel 626 can include and/or be a tubular channel, such as the flush lumen 226 of FIG. 7 and/or the flush lumen 326 of FIGS. 9A-9D.

FIG. 12A additionally shows a configuration in which a second reservoir 640 is coupled to the fluid inlet port 668 of the (first) reservoir 660 via the stopcock 632 (e.g., with a luer fitting).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a handle comprising a housing; a reservoir external to the housing, wherein the reservoir is filled with fluid and has an adjustable fluid volume; and a shaft extending within and distally from the handle and having a main lumen, wherein the main lumen is fluidly coupled to the reservoir by a channel disposed within the handle, wherein the reservoir is configured to passively supply fluid to the main lumen based on a fluid pressure in the main lumen.

Example 2. The delivery apparatus of any example herein, particularly example 1, wherein the reservoir is a compressible reservoir.

Example 3. The delivery apparatus of any example herein, particularly any one of examples 1-2, wherein the reservoir comprises an outer wall that comprises a flexible material, and wherein the reservoir is configured to compress as its fluid volume reduces.

Example 4. The delivery apparatus of any example herein, particularly example 3, wherein the reservoir comprises an inner frame disposed interior of the outer wall, wherein the inner frame defines a compressed volume of the reservoir, and wherein the inner frame is configured to restrict the fluid volume of the reservoir to be at least equal to the compressed volume.

Example 5. The delivery apparatus of any example herein, particularly example 4, wherein the reservoir is configured to transition between an expanded configuration, in which the fluid volume of the reservoir is an expanded volume that is greater than the compressed volume, and a compressed configuration, in which the fluid volume of the reservoir is equal to the compressed volume.

Example 6. The delivery apparatus of any example herein, particularly any one of examples 4-5, wherein the outer wall engages the inner frame when the reservoir is in the compressed configuration.

Example 7. The delivery apparatus of any example herein, particularly any one of examples 4-6, wherein the inner frame is configured to permit the fluid to flow through the inner frame as the reservoir transitions between the expanded configuration and the compressed configuration.

Example 8. The delivery apparatus of any example herein, particularly any one of examples 4-7, wherein the inner frame comprises a rigid material.

Example 9. The delivery apparatus of any example herein, particularly any one of examples 4-8, wherein the inner frame comprises one or more of a mesh structure, a perforated structure, and a plurality of spaced-apart frame members.

Example 10. The delivery apparatus of any example herein, particularly any one of examples 1-9, wherein the handle further comprises a flush port coupled to the housing, and wherein the flush port is fluidly coupled to the reservoir.

Example 11. The delivery apparatus of any example herein, particularly example 10, wherein the reservoir comprises a fluid outlet port, and wherein the delivery apparatus comprises a fluid conduit that fluidly couples the fluid outlet port to the flush port.

Example 12. The delivery apparatus of any example herein, particularly example 11, wherein the fluid conduit terminates at a fluid conduit connector that is configured to engage the flush port to fluidly couple the reservoir to the flush port.

Example 13. The delivery apparatus of any example herein, particularly any one of examples 11-12, wherein the fluid conduit comprises a flexible conduit.

Example 14. The delivery apparatus of any example herein, particularly any one of examples 1-13, wherein the reservoir is a first reservoir, and wherein the delivery apparatus further comprises a second reservoir fluidly connected to the first reservoir.

Example 15. The delivery apparatus of any example herein, particularly example 14, wherein the second reservoir is fluidly connected to the main lumen via the first reservoir.

Example 16. The delivery apparatus of any example herein, particularly any one of examples 14-15, wherein the first reservoir comprises a fluid inlet port, and wherein the second reservoir is coupled to the fluid inlet port.

Example 17. The delivery apparatus of any example herein, particularly any one of examples 14-16, wherein the handle further comprises a flush port coupled to the housing, wherein the reservoir comprises a fluid outlet port, wherein the delivery apparatus comprises a fluid conduit that fluidly couples the fluid outlet port to the flush port with a first connector type, and wherein the second reservoir is coupled to a fluid inlet port of the first reservoir with a second connector type that is different from the first connector type.

Example 18. The delivery apparatus of any example herein, particularly example 17, wherein the second reservoir is fluidly connected to the first reservoir via a stopcock.

Example 19. The delivery apparatus of any example herein, particularly any one of examples 17-18, wherein the handle further comprises a seal housing assembly including one or more fluid seals, and wherein the flush port is disposed at a proximal end of the reservoir that is disposed adjacent to the seal housing assembly.

Example 20. The delivery apparatus of any example herein, particularly any one of examples 17-19, wherein the channel extends from the flush port and radially inward to the main lumen.

Example 21. The delivery apparatus of any example herein, particularly any one of examples 14-18, wherein the second reservoir comprises a barrel and a movable wall disposed within the barrel, the barrel and the movable wall defining a second reservoir cavity that contains the fluid therein, and wherein the movable wall is configured to be selectively moved to drive fluid from the second reservoir to the first reservoir.

Example 22. The delivery apparatus of any example herein, particularly any one of examples 14-21, wherein the second reservoir comprises a syringe.

Example 23. The delivery apparatus of any example herein, particularly any one of examples 1-22, further comprising a reservoir mount configured to support the reservoir relative to the handle.

Example 24. The delivery apparatus of any example herein, particularly example 23, wherein the reservoir mount is configured to maintain the reservoir in a fixed position relative to the handle.

Example 25. The delivery apparatus of any example herein, particularly any one of examples 23-24, wherein the handle is configured to be supported relative to a delivery apparatus preparation surface by a handle support structure, and wherein the reservoir mount is configured to be coupled to the delivery apparatus preparation surface.

Example 26. The delivery apparatus of any example herein, particularly example 25, wherein the reservoir mount is configured to be one or both of fixedly coupled to the delivery apparatus preparation surface and removably coupled to the delivery apparatus preparation surface.

Example 27. The delivery apparatus of any example herein, particularly any one of examples 23-26, wherein the reservoir mount is configured to be one or both of fixedly coupled to the reservoir and removably coupled to the reservoir.

Example 28. The delivery apparatus of any example herein, particularly any one of examples 1-27, wherein the reservoir is configured to be supported by a delivery apparatus preparation surface at least substantially underneath the handle.

Example 29. The delivery apparatus of any example herein, particularly any one of examples 1-28, wherein the reservoir is at least partially supported by the handle.

Example 30. The delivery apparatus of any example herein, particularly any one of examples 1-29, wherein the reservoir extends at least substantially around a circumference of the handle.

Example 31. The delivery apparatus of any example herein, particularly any one of examples 1-30, wherein the channel is an annular channel that extends at least substantially circumferentially around the main lumen.

Example 32. The delivery apparatus of any example herein, particularly any one of examples 1-31, wherein the reservoir is fluidly sealed against the handle by one or more reservoir seals.

Example 33. The delivery apparatus of any example herein, particularly example 32, wherein the one or more reservoir seals comprise annular seals positioned on opposite sides of the reservoir.

Example 34. The delivery apparatus of any example herein, particularly any one of examples 1-33, wherein the handle further comprises a seal housing assembly including one or more fluid seals, and wherein the channel is disposed at a proximal end of the reservoir that is disposed adjacent to the seal housing assembly.

Example 35. The delivery apparatus of any example herein, particularly any one of examples 1-34, wherein the handle further comprises a rotatable knob operatively coupled to flex mechanisms configured to adjust a curvature of a distal end portion of the shaft.

Example 36. The delivery apparatus of any example herein, particularly any one of examples 1-35, wherein the fluid inside the reservoir is saline.

Example 37. A delivery assembly comprising: an implant catheter; and a guide catheter comprising: a handle comprising a housing and a flush port coupled to the housing; and a shaft extending distally from within the handle and having a main lumen configured to receive a portion of the implant catheter therethrough; and a reservoir disposed external to the housing, wherein the reservoir is filled with fluid, wherein the main lumen is fluidly coupled to the reservoir via the flush port.

Example 38. The delivery assembly of any example herein, particularly example 37, wherein the reservoir is a compressible reservoir with an adjustable fluid volume.

Example 39. The delivery assembly of any example herein, particularly any one of examples 37-38, wherein the reservoir comprises an outer wall and an inner frame disposed interior of the outer wall, wherein the inner frame defines a compressed volume of the reservoir, and wherein the inner frame is configured to restrict a fluid volume of the reservoir to be at least equal to the compressed volume.

Example 40. The delivery assembly of any example herein, particularly example 39, wherein the reservoir is configured to transition between an expanded configuration, in which the fluid volume of the reservoir is an expanded volume that is greater than the compressed volume, and a compressed configuration, in which the fluid volume of the reservoir is equal to the compressed volume.

Example 41. The delivery assembly of any example herein, particularly example 40, wherein the outer wall engages the inner frame when the reservoir is in the compressed configuration.

Example 42. The delivery assembly of any example herein, particularly any one of examples 40-41, wherein the inner frame is configured to permit the fluid to flow through the inner frame as the reservoir transitions between the expanded configuration and the compressed configuration.

Example 43. The delivery assembly of any example herein, particularly any one of examples 39-42, wherein the inner frame comprises a rigid material.

Example 44. The delivery assembly of any example herein, particularly any one of examples 39-43, wherein the inner frame comprises one or more of a mesh structure, a perforated structure, and a plurality of spaced-apart frame members.

Example 45. The delivery assembly of any example herein, particularly any one of examples 37-44, wherein the main lumen is fluidly coupled to the reservoir by a channel extending at least partially between the main lumen and the reservoir.

Example 46. The delivery assembly of any example herein, particularly example 45, wherein the handle further comprises a seal housing assembly including one or more fluid seals, and wherein the sealing housing assembly is disposed at a proximal end of handle.

Example 47. The delivery assembly of any example herein, particularly example 46, wherein the channel is disposed adjacent to the seal housing assembly.

Example 48. The delivery assembly of any example herein, particularly any one of examples 37-47, wherein the reservoir comprises a fluid outlet port, and wherein the delivery assembly comprises a fluid conduit that fluidly couples the fluid outlet port to the flush port.

Example 49. The delivery assembly of any example herein, particularly example 48, wherein the fluid conduit terminates at a fluid conduit connector that is configured to engage the flush port to fluidly couple the reservoir to the flush port.

Example 50. The delivery assembly of any example herein, particularly any one of examples 48-49, wherein the fluid conduit comprises a flexible conduit.

Example 51. The delivery assembly of any example herein, particularly any one of examples 37-50, wherein the reservoir is a first reservoir, and wherein the delivery assembly further comprises a second reservoir fluidly connected to the first reservoir.

Example 52. The delivery assembly of any example herein, particularly example 51, wherein the second reservoir is fluidly connected to the main lumen via the first reservoir.

Example 53. The delivery assembly of any example herein, particularly any one of examples 51-52, wherein the first reservoir comprises a fluid inlet port, and wherein the second reservoir is coupled to the fluid inlet port.

Example 54. The delivery assembly of any example herein, particularly example 53, wherein the reservoir comprises a fluid outlet port, wherein the delivery assembly comprises a fluid conduit that fluidly couples the fluid outlet port to the flush port with a first connector type, and wherein the second reservoir is coupled to the fluid inlet port with a second connector type that is different from the first connector type.

Example 55. The delivery assembly of any example herein, particularly any one of examples 51-54, wherein the second reservoir is fluidly connected to the first reservoir via a stopcock.

Example 56. The delivery assembly of any example herein, particularly any one of examples 51-55, wherein the second reservoir comprises a barrel and a movable wall disposed within the barrel, the barrel and the movable wall defining a second reservoir cavity that contains the fluid therein, and wherein the movable wall is configured to be selectively moved to drive fluid from the second reservoir to the first reservoir.

Example 57. The delivery assembly of any example herein, particularly any one of examples 51-56, wherein the second reservoir comprises a syringe.

Example 58. The delivery assembly of any example herein, particularly any one of examples 37-57, further comprising a reservoir mount configured to support the reservoir relative to the handle.

Example 59. The delivery assembly of any example herein, particularly example 58, wherein the reservoir mount is configured to maintain the reservoir in a fixed position relative to the handle.

Example 60. The delivery assembly of any example herein, particularly any one of examples 58-59, wherein the handle is configured to be supported relative to a delivery apparatus preparation surface by a handle support structure, and wherein the reservoir mount is configured to be coupled to the delivery apparatus preparation surface.

Example 61. The delivery assembly of any example herein, particularly any one of examples 58-60, wherein the reservoir mount is configured to be one or both of fixedly coupled to the delivery apparatus preparation surface and removably coupled to the delivery apparatus preparation surface.

Example 62. The delivery assembly of any example herein, particularly any one of examples 58-61, wherein the reservoir mount is configured to be one or both of fixedly coupled to the reservoir and removably coupled to the reservoir.

Example 63. The delivery assembly of any example herein, particularly any one of examples 37-62, wherein the reservoir is configured to be supported by a delivery apparatus preparation surface at least substantially underneath the handle.

Example 64. The delivery assembly of any example herein, particularly any one of examples 37-63, wherein the reservoir is at least partially supported by the handle.

Example 65. The delivery assembly of any example herein, particularly any one of examples 37-64, wherein the reservoir extends at least substantially around a circumference of the handle.

Example 66. The delivery assembly of any example herein, particularly any one of examples 37-65, wherein the channel is an annular channel that extends at least substantially circumferentially around the main lumen.

Example 67. The delivery assembly of any example herein, particularly any one of examples 37-66, wherein the reservoir is fluidly sealed against the handle by one or more reservoir seals.

Example 68. The delivery assembly of any example herein, particularly example 67, wherein the one or more reservoir seals comprise annular seals positioned on opposite sides of the reservoir.

Example 69. The delivery assembly of any example herein, particularly any one of examples 37-68, wherein the handle further comprises a flex mechanism disposed within the housing and surrounding a portion of the shaft, and wherein the flex mechanism is configured to adjust a curvature of a distal end portion of the shaft.

Example 70. The delivery assembly of any example herein, particularly example 69, wherein the handle further comprises a seal housing assembly including one or more fluid seals and a rotatable knob operatively coupled to the flex mechanism, and wherein the reservoir is disposed between, in an axial direction, the knob and the seal housing assembly.

Example 71. A guide sheath system comprising: a handle comprising: a housing and a flush port coupled to the housing; and a seal housing assembly including one or more fluid seals; and a compressible reservoir disposed external to the housing, wherein the reservoir is filled with fluid and has an adjustable volume, and wherein the flush port is fluidly coupled to the reservoir; and a shaft extending within and distally from the handle and having a main lumen that extends within the housing and through the seal housing assembly, wherein the main lumen is fluidly coupled to the reservoir.

Example 72. The guide sheath system of any example herein, particularly example 71, wherein the reservoir comprises a flexible wall and a cavity defined by the wall, and wherein the cavity is filled with the fluid.

Example 73. The guide sheath system of any example herein, particularly any one of examples 71-72, wherein the reservoir comprises an outer wall and an inner frame disposed interior of the outer wall, wherein the inner frame defines a compressed volume of the reservoir, and wherein the inner frame is configured to restrict a fluid volume of the reservoir to be at least equal to the compressed volume.

Example 74. The guide sheath system of any example herein, particularly example 73, wherein the reservoir is configured to transition between an expanded configuration, in which the fluid volume of the reservoir is an expanded volume that is greater than the compressed volume, and a compressed configuration, in which the fluid volume of the reservoir is equal to the compressed volume.

Example 75. The guide sheath system of any example herein, particularly example 74, wherein the outer wall engages the inner frame when the reservoir is in the compressed configuration.

Example 76. The guide sheath system of any example herein, particularly any one of examples 74-75, wherein the inner frame is configured to permit the fluid to flow through the inner frame as the reservoir transitions between the expanded configuration and the compressed configuration.

Example 77. The guide sheath system of any example herein, particularly any one of examples 73-76, wherein the inner frame comprises a rigid material.

Example 78. The guide sheath system of any example herein, particularly any one of examples 73-77, wherein the inner frame comprises one or more of a mesh structure, a perforated structure, and a plurality of spaced-apart frame members.

Example 79. The guide sheath system of any example herein, particularly any one of examples 71-78, wherein the handle comprises a fluid inlet configured as a first channel that extends between the flush port and the main lumen, and wherein the reservoir comprises a fluid outlet port and a fluid conduit configured as a second channel that extends between the fluid outlet port and the fluid inlet.

Example 80. The guide sheath system of any example herein, particularly example 79, wherein the first channel is disposed adjacent to the seal housing assembly.

Example 81. The guide sheath system of any example herein, particularly any one of examples 71-80, wherein the reservoir comprises a fluid outlet port, and wherein the guide sheath system comprises a fluid conduit that fluidly couples the fluid outlet port to the flush port.

Example 82. The guide sheath system of any example herein, particularly example 81, wherein the fluid conduit terminates at a fluid conduit connector that is configured to engage the flush port to fluidly couple the reservoir to the flush port.

Example 83. The guide sheath system of any example herein, particularly any one of examples 81-82, wherein the fluid conduit comprises a flexible conduit.

Example 84. The guide sheath system of any example herein, particularly any one of examples 71-83, wherein the reservoir is a first reservoir, and wherein the guide sheath system further comprises a second reservoir fluidly connected to the first reservoir.

Example 85. The guide sheath system of any example herein, particularly example 84, wherein the second reservoir is fluidly connected to the main lumen via the first reservoir.

Example 86. The guide sheath system of any example herein, particularly any one of examples 84-85, wherein the first reservoir comprises a fluid inlet port, and wherein the second reservoir is coupled to the fluid inlet port.

Example 87. The guide sheath system of any example herein, particularly example 86, wherein the first reservoir comprises a fluid outlet port, wherein the guide sheath system comprises a fluid conduit that fluidly couples the fluid outlet port to the flush port with a first connector type, and wherein the second reservoir is coupled to the fluid inlet port with a second connector type that is different from the first connector type.

Example 88. The guide sheath system of any example herein, particularly any one of examples 84-86, wherein the second reservoir is fluidly connected to the first reservoir via a stopcock.

Example 89. The guide sheath system of any example herein, particularly any one of examples 84-89, wherein the second reservoir comprises a barrel and a movable wall disposed within the barrel, the barrel and the movable wall defining a second reservoir cavity that contains the fluid therein, and wherein the movable wall is configured to be selectively moved to drive fluid from the second reservoir to the first reservoir.

Example 90. The guide sheath system of any example herein, particularly any one of examples 84-89, wherein the second reservoir comprises a syringe.

Example 91. The guide sheath system of any example herein, particularly any one of examples 71-90, further comprising a reservoir mount configured to support the reservoir relative to the handle.

Example 92. The guide sheath system of any example herein, particularly example 91, wherein the reservoir mount is configured to maintain the reservoir in a fixed position relative to the handle.

Example 93. The guide sheath system of any example herein, particularly any one of examples 91-92, wherein the handle is configured to be supported relative to a delivery apparatus preparation surface by a handle support structure, and wherein the reservoir mount is configured to be coupled to the delivery apparatus preparation surface.

Example 94. The guide sheath system of any example herein, particularly any one of examples 91-93, wherein the reservoir mount is configured to be one or both of fixedly coupled to the delivery apparatus preparation surface and removably coupled to the delivery apparatus preparation surface.

Example 95. The guide sheath system of any example herein, particularly any one of examples 91-94, wherein the reservoir mount is configured to be one or both of fixedly coupled to the reservoir and removably coupled to the reservoir.

Example 96. The guide sheath system of any example herein, particularly any one of examples 71-95, wherein the reservoir is configured to be supported by a delivery apparatus preparation surface at least substantially underneath the handle.

Example 97. The guide sheath system of any example herein, particularly any one of examples 71-96, wherein the reservoir is at least partially supported by the handle.

Example 98. The guide sheath system of any example herein, particularly any one of examples 71-97, wherein the reservoir extends at least substantially around a circumference of the handle.

Example 99. The guide sheath system of any example herein, particularly any one of examples 71-98, wherein the reservoir is fluidly coupled to the main lumen via an annular channel that extends at least substantially circumferentially around the main lumen.

Example 100.The guide sheath system of any example herein, particularly any one of examples 71-99, wherein the reservoir is fluidly sealed against the handle by one or more reservoir seals.

Example 101.The guide sheath system of any example herein, particularly example 100, wherein the one or more reservoir seals comprise annular seals positioned on opposite sides of the reservoir.

Example 102.The guide sheath system of any example herein, particularly any one of examples 71-101, wherein the handle further comprises a flex mechanism that is configured to adjust a curvature of a distal end portion of the shaft and disposed within the housing, and wherein the flex mechanism is disposed distal to the seal housing assembly.

Example 103.The guide sheath system of any example herein, particularly example 102, wherein the handle further comprises a rotatable knob operatively coupled to the flex mechanism.

Example 104.A method for implanting a prosthetic medical device, comprising: inserting a shaft of a guide catheter into a vessel of a patient, the shaft having a main lumen and extending from within and distal to a handle of the guide catheter; inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter; and compressing a fluid reservoir disposed external to a housing of the handle and flowing fluid from within the fluid reservoir into the main lumen such that a volume of the fluid reservoir decreases.

Example 105.The method of any example herein, particularly example 104, wherein the compressing the fluid reservoir occurs passively as fluid from within the fluid reservoir is pulled into the main lumen by decreasing fluid pressure within the main lumen as the distal end portion of the first implant catheter is advanced further along the main lumen.

Example 106.The method any one of any example herein, particularly examples 104-105, wherein the compressing the fluid reservoir includes contracting an external wall of the fluid reservoir inward such that a fluid volume of an internal cavity of the fluid reservoir that contains the fluid decreases as the fluid flows into the main lumen.

Example 107.The method of any example herein, particularly example 106, wherein the fluid reservoir comprises an inner frame disposed interior of the external wall, wherein the inner frame defines a compressed volume of the fluid reservoir, and wherein the inner frame is configured to restrict the fluid volume of the reservoir to be at least equal to the compressed volume.

Example 108.The method of any example herein, particularly example 107, wherein the reservoir is configured to transition between an expanded configuration, in which the fluid volume of the fluid reservoir is an expanded volume that is greater than the compressed volume, and a compressed configuration, in which the fluid volume of the fluid reservoir is equal to the compressed volume, and wherein the flowing the fluid from within the fluid reservoir into the main lumen comprises flowing such that the fluid volume of the fluid reservoir reaches the compressed volume.

Example 109.The method of any example herein, particularly any one of examples 104-108, wherein flowing fluid from within the fluid reservoir into the main lumen includes flowing fluid from within the fluid reservoir through a fluid channel extending between the fluid reservoir and the main lumen inside the housing.

Example 110.The method of any example herein, particularly any one of examples 104-109, wherein the handle includes a flush port coupled to the housing and disposed distal to one or more fluid seals of the handle that are disposed adjacent to the proximal end of the guide catheter, and wherein the fluid reservoir is fluidly coupled to the flush port.

Example 111.The method of any example herein, particularly any one of examples 104-110, wherein the fluid reservoir is a first fluid reservoir, and wherein the method further comprises flowing fluid from a second fluid reservoir to the first fluid reservoir to drive fluid from the first fluid reservoir to the main lumen.

Example 112.The method of any example herein, particularly example 111, wherein the first fluid reservoir comprises a fluid inlet port, and wherein the second fluid reservoir is fluidly connected to the fluid inlet port.

Example 113.The method of any example herein, particularly any one of examples 111-112, wherein the second fluid reservoir is connected to a fluid inlet port of the first fluid reservoir via a stopcock, and wherein the method further comprises, subsequent to flowing the fluid from the second fluid reservoir to the first fluid reservoir, transitioning the stopcock to a closed position.

Example 114.The method of any example herein, particularly any one of examples 104-113, further comprising implanting the prosthetic medical device at the target implantation site, removing the first implant catheter from the guide catheter, and inserting a second implant catheter into the guide catheter and advancing the second implant catheter through the main lumen toward the target implantation site.

Example 115.The method of any example herein, particularly example 114, wherein the first implant catheter is a docking device delivery apparatus and the prosthetic medical device is a docking device, and wherein the second implant catheter is a prosthetic heart valve delivery apparatus configured to deliver a prosthetic heart valve within the implanted docking device.

Example 116.A method for implanting a prosthetic medical device, comprising: inserting a shaft of a guide catheter into a vessel of a patient, the shaft having a main lumen and extending from within and distal to a handle of the guide catheter; and inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter, wherein, as the distal end portion of the first implant catheter is advanced through the main lumen, fluid is pulled from a fluid reservoir into the main lumen such that a volume of the fluid reservoir decreases, and wherein the fluid reservoir is disposed external to the handle.

Example 117.The method of any example herein, particularly example 116, wherein as fluid is pulled from the fluid reservoir into the main lumen, a wall of the fluid reservoir contracts inward and a fluid cavity containing the fluid and defined by the wall of the fluid reservoir decreases in size.

Example 118.The method of any example herein, particularly any one of examples 116-117, wherein the fluid reservoir is fluidly coupled to the main lumen by a channel disposed within the handle.

Example 119.The method of any example herein, particularly any one of examples 116-118, wherein the handle includes a flush port coupled to a housing of the handle and disposed distal to one or more fluid seals of the handle that are disposed adjacent to the proximal end of the guide catheter, and wherein the fluid reservoir is fluidly coupled to the flush port.

Example 120.The method of any example herein, particularly any one of examples 116-119, wherein the fluid reservoir is a first fluid reservoir, and wherein the method further comprises flowing fluid from a second fluid reservoir to the first fluid reservoir to drive fluid from the first fluid reservoir to the main lumen.

Example 121.The method of any example herein, particularly example 120, wherein the first fluid reservoir comprises a fluid inlet port, and wherein the second fluid reservoir is fluidly connected to the fluid inlet port.

Example 122.The method of any example herein, particularly any one of examples 120-121, wherein the second fluid reservoir is connected to a fluid inlet port of the first fluid reservoir via a stopcock, and wherein the method further comprises, subsequent to flowing the fluid from the second fluid reservoir to the first fluid reservoir, transitioning the stopcock to a closed position.

Example 123.The method of any example herein, particularly any one of examples 116-122, further comprising implanting the prosthetic medical device at the target implantation site, removing the first implant catheter from the guide catheter, and inserting a second implant catheter into the guide catheter and advancing the second implant catheter through the main lumen toward the target implantation site.

Example 124.The method of any example herein, particularly example 123, wherein the first implant catheter is a docking device delivery apparatus and the prosthetic medical device is a docking device, and wherein the second implant catheter is a prosthetic heart valve delivery apparatus configured to deliver a prosthetic heart valve within the implanted docking device.

Example 125.The method of any example herein, particularly any one of examples 104-124, wherein the method is performed on a living animal or on a non-living simulation.

Example 126.A method comprising sterilizing the prosthetic heart valve, apparatus, and/or assembly of any example herein.

Example 127.A prosthetic heart valve, apparatus, or assembly of any example herein, particularly any one of examples 1-103, wherein the prosthetic heart valve, apparatus, or assembly is sterilized.

The features described herein with regard to any example can be combined with other features described in any one or more of the other examples, unless otherwise stated. For example, any one or more of the features of one guide catheter can be combined with any one or more features of another guide catheter. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus comprising:
a handle comprising a housing;
a reservoir external to the housing, wherein the reservoir is filled with fluid and has an adjustable fluid volume; and
a shaft extending within and distally from the handle and having a main lumen, wherein the main lumen is fluidly coupled to the reservoir by a channel disposed within the handle,
wherein the reservoir is configured to passively supply fluid to the main lumen based on a fluid pressure in the main lumen.

2. The delivery apparatus of claim 1, wherein the reservoir comprises an outer wall that comprises a flexible material, and wherein the reservoir is configured to compress as its fluid volume reduces.

3. The delivery apparatus of claim 1, wherein the handle further comprises a flush port coupled to the housing, wherein the flush port is fluidly coupled to the reservoir, wherein the reservoir comprises a fluid outlet port, and wherein the delivery apparatus comprises a fluid conduit that fluidly couples the fluid outlet port to the flush port.

4. The delivery apparatus of claim 3, wherein the fluid conduit comprises a flexible conduit.

5. The delivery apparatus of claim 1, wherein the reservoir is a first reservoir, and wherein the delivery apparatus further comprises a second reservoir fluidly connected to the first reservoir.

6. The delivery apparatus of claim 5, wherein the second reservoir is fluidly connected to the main lumen via the first reservoir.

7. The delivery apparatus of claim 5, wherein the handle further comprises a flush port coupled to the housing, wherein the reservoir comprises a fluid outlet port, wherein the delivery apparatus comprises a fluid conduit that fluidly couples the fluid outlet port to the flush port with a first connector type, and wherein the second reservoir is coupled to a fluid inlet port of the first reservoir with a second connector type that is different from the first connector type.

8. The delivery apparatus of claim 5, wherein the second reservoir comprises a barrel and a movable wall disposed within the barrel, the barrel and the movable wall defining a second reservoir cavity that contains the fluid therein, and wherein the movable wall is configured to be selectively moved to drive fluid from the second reservoir to the first reservoir.

9. A delivery assembly comprising:
an implant catheter; and
a guide catheter comprising:
a handle comprising a housing and a flush port coupled to the housing; and
a shaft extending distally from within the handle and having a main lumen configured to receive a portion of the implant catheter therethrough; and
a reservoir disposed external to the housing, wherein the reservoir is filled with fluid,
wherein the main lumen is fluidly coupled to the reservoir via the flush port.

10. The delivery assembly of claim 9, wherein the reservoir is a compressible reservoir with an adjustable fluid volume.

11. The delivery assembly of clam 9, wherein the reservoir comprises an outer wall and an inner frame disposed interior of the outer wall, wherein the inner frame defines a compressed volume of the reservoir, and wherein the inner frame is configured to restrict a fluid volume of the reservoir to be at least equal to the compressed volume.

12. The delivery assembly of claim 11, wherein the reservoir is configured to transition between an expanded configuration, in which the fluid volume of the reservoir is an expanded volume that is greater than the compressed volume, and a compressed configuration, in which the fluid volume of the reservoir is equal to the compressed volume, and wherein the inner frame is configured to permit the fluid to flow through the inner frame as the reservoir transitions between the expanded configuration and the compressed configuration.

13. The delivery assembly of claim 11, wherein the inner frame comprises a rigid material.

14. The delivery assembly of claim 9, wherein the reservoir is a first reservoir, and wherein the delivery assembly further comprises a second reservoir fluidly connected to the first reservoir via a stopcock.

15. A method for implanting a prosthetic medical device, comprising:
inserting a shaft of a guide catheter into a vessel of a patient, the shaft having a main lumen and extending from within and distal to a handle of the guide catheter;
inserting a distal end portion of a first implant catheter into a proximal end of the guide catheter and advancing the distal end portion of the first implant catheter through the main lumen of the guide catheter toward a target implantation site for a prosthetic medical device mounted on the distal end portion of the first implant catheter; and
compressing a fluid reservoir disposed external to a housing of the handle and flowing fluid from within the fluid reservoir into the main lumen such that a volume of the fluid reservoir decreases.

16. The method of claim 15, wherein the compressing the fluid reservoir occurs passively as fluid from within the fluid reservoir is pulled into the main lumen by decreasing fluid pressure within the main lumen as the distal end portion of the first implant catheter is advanced further along the main lumen.

17. The method of claim 15, wherein the compressing the fluid reservoir includes contracting an external wall of the fluid reservoir inward such that a fluid volume of an internal cavity of the fluid reservoir that contains the fluid decreases as the fluid flows into the main lumen.

18. The method of claim 15, wherein the fluid reservoir is a first fluid reservoir, and wherein the method further comprises flowing fluid from a second fluid reservoir to the first fluid reservoir to drive fluid from the first fluid reservoir to the main lumen.

19. The method of claim 18, wherein the second fluid reservoir is connected to a fluid inlet port of the first fluid reservoir via a stopcock, and wherein the method further comprises, subsequent to flowing the fluid from the second fluid reservoir to the first fluid reservoir, transitioning the stopcock to a closed position.

20. The method of claim 15, further comprising implanting the prosthetic medical device at the target implantation site, removing the first implant catheter from the guide catheter, and inserting a second implant catheter into the guide catheter and advancing the second implant catheter through the main lumen toward the target implantation site.
